(19) United States
(12) Patent Application Publication
(10) Pub. No.: US 2024/0189432 A1
(43) Pub. Date: Jun. 13, 2024

(54) POLYOXAZOLINE-BOUND ALBUMIN, ARTIFICIAL PLASMA EXPANDER, AND HEMORRHAGIC SHOCK RESUSCITATION FLUID

(71) Applicant: CHUO UNIVERSITY, Hachioji-shi, Tokyo (JP)

(72) Inventors: Teruyuki KOMATSU, Hachioji-shi, Tokyo (JP); Wataru OKAMOTO, Hachioji-shi, Tokyo (JP); Tomone USUI, Hachioji-shi, Tokyo (JP)

(73) Assignee: CHUO UNIVERSITY, Hachioji-shi, Tokyo (JP)

(21) Appl. No.: 18/547,504

(22) PCT Filed: Feb. 18, 2022
(86) PCT No.: PCT/JP2022/006785

§ 371 Date: Aug. 23, 2023

(30) Foreign Application Priority Data
Feb. 24, 2021 (JP) 2021-027892

(51) Int. Cl. A61K 47/59; A61K 38/38; A61K 47/54; A61K 47/69; A61P 7/08

(52) U.S. Cl. CPC A61K 47/595; A61K 38/38; A61K 47/545; A61K 47/6925; A61P 7/08

(57) ABSTRACT

It could be helpful to provide albumin to which a water-soluble polymer that has high biocompatibility and is easily prepared (synthesized) is bound; and an artificial plasma expander and a hemorrhagic shock resuscitation fluid that each contain the water-soluble polymer-bound albumin. The solution is a polyoxazoline-bound albumin (100) including an albumin (10) as a core and a polyoxazoline (20) as a shell, which is covalently bound to the albumin (10) via a cross-linker; and an artificial plasma expander and a hemorrhagic shock resuscitation fluid that each contain the polyoxazoline-bound albumin (100).

### TECHNICAL FIELD

This disclosure relates to a polyoxazoline-bound albumin, an artificial plasma expander, and a hemorrhagic shock resuscitation fluid.

### BACKGROUND

Serum albumin is a simple protein, which accounts for about 60% of plasma proteins, and takes a role in maintaining a colloid osmotic pressure and storing and transporting various endogenous substances (metabolite, hormone, etc.) and exogenous substances (drug, etc.) in blood flow. The albumin separated from donated blood (plasma fractionation) and purified is formulated and widely used in clinical practice. The purpose of administering the albumin preparation is to maintain the colloid osmotic pressure and ensure the circulating blood volume (circulating plasma volume). Specifically, in a case where hypoalbuminemia develops due to bleeding, an increase in permeability of the capillary, a decrease in albumin synthesis in the liver, excess excretion from the kidney or bowels, facilitation of metabolism, dilution by intraoperative infusion, or the like, the albumin preparation is administered. The albumin preparation includes two types: an isotonic albumin preparation and a hypertonic albumin preparation. The isotonic albumin preparation is used in emergency hemorrhagic shock due to injury, sepsis, cardiac surgery using a heart-lung machine, extracorporeal circulation with unstable hemodynamics, severe burn, and pathological conditions such as pregnancy-induced hypertension. The hypertonic albumin preparation can increase the colloid osmotic pressure to draw water into the blood vessel, thus being used in hemorrhagic shock, and pathological conditions such as refractory ascites associated with hepatic cirrhosis, refractory edema, and nephrotic syndrome associated with pulmonary edema. The albumin preparation allows viral inactivation by heating, and there is no risk of viral infection. However, the degree of self-sufficiency of the albumin preparation in Japan is low, 64% (2018), and “Domestic self-sufficiency” that is the basic philosophy in Blood Law has not been achieved. There is concern that the degree of self-sufficiency will further decrease as the population ages and fewer babies are born, the number of elderly people in need of blood transfusions increases, and the number of blood donors (young people) declines.

As an alternative of the albumin preparation, that is, an artificial plasma expander and a hemorrhagic shock resuscitation fluid, a preparation using polysaccharide has been developed since the 1970s, and a low molecular dextran preparation and a hydroxyethyl starch (HES) preparation are currently in practical use (see, for example, Patent Literatures (PTLs) 1 and 2). However, when the HES preparation is administered, side effects such as blood coagulation disorder, renal dysfunction, shock, and amylase elevation may occur. Against this background, there are currently high hopes for the development of new artificial plasma expander and hemorrhagic shock resuscitation fluid.

On the other hand, Japan is a pet-owning country with more than 18.13 million dogs and cats, far more than the population of children under years old (15.11 million). Pets are also aging, and the demand for animal medical care continues to grow year by year. However, no sufficient system is ready for blood transfusion treatment, as there is not animal blood bank in the first place. Naturally, there are no animal albumin preparations as plasma derivatives (for example, a canine serum albumin preparation separated and purified from canine blood, or a feline serum albumin preparation separated and purified from feline blood). As a treatment for an animal with hypoalbuminemia, there is a method of obtaining and transfusing the plasma of the animal, but it is difficult to stably ensure the plasma. Although the HES preparation is reluctantly used, the above-described side effects occur, and there is also a drawback that the retention time in blood is short. Against this background, there are currently high hopes for the development of an artificial plasma expander for animal that is safer and has longer retentivity in blood.

Polyethylene glycol (PEG) is a water-soluble synthetic polymer with excellent biocompatibility. Even in the case of a heteroprotein, if its molecular surface is coated with PEG, immunological stealth is rendered. However, it is reported that a serum antibody against PEG is produced in the body of the patient treated with PEG-bound asparaginase or PEG-bound uricase. In the presence of an anti-PEG antibody, the administered PEG-bound preparation is rapidly excreted from the body (see, for example, Non-patent Literatures (NPLs) 1 and 2). Further, there is a report that patients who have not been treated with the PEG-bound preparation also have the anti-PEG antibody at a rate of 25% or more (for example, NPLs 3 and 4). This may be due to the fact that PEG is used in various products on the market, including foods and cosmetics. It has been also revealed that cell vacuolation is observed when PEG-bound hemoglobin is repeatedly administered (for example, NPL 5). Under these circumstances, attention is focused on the development of a new water-soluble polymer with biocompatibility that can replace PEG.

### CITATION LIST

### Patent Literature

PTL 1: JP H06-133791A

PTL 2: JP 2007-525588A

### Non-patent Literature

NPL 1: J. K. Armstrong et al., Cancer, 2007, 110, 103-111.

NPL 2: M. R. Sherman et al., Adv. Drug Delivery Rev., 2008, 6, 59-68.

NPL 3: R. M. Leger et al., Transfusion, 2001, 41, 29S.

NPL 4: C. Lubich et al., Pharm. Res., 2016, 33, 2239-2249.

NPL 5: C. Conover et al., Artif. Cells, Blood Subs., Immob. Biotechnol., 1996, 24, 599-611.

### SUMMARY

### Technical Problem

The safe use of a preparation made of easily available heterologous albumin as an artificial plasma expander and a hemorrhagic shock resuscitation fluid for human and for animal would result in a major contribution to not only general medical care but also animal medical care. One of the method is to bind PEG to the surface of heterologous albumin to render immunological stealth, but the production of PEG antibodies is a concern. Therefore, it is strongly desired to develop an artificial plasma expander and a hemorrhagic shock resuscitation fluid, which are made of albumin to which a water-soluble polymer that has high biocompatibility (for example, no renal excretion, and no side effects such as renal dysfunction and antigen-antibody reaction) and easily prepared (synthesized) is bound, instead of PEG.

It could be helpful to provide albumin to which a water-soluble polymer that has high biocompatibility and is easily prepared (synthesized) is bound, and an artificial plasma expander and a hemorrhagic shock resuscitation fluid that use the albumin as their active ingredients.

### Solution to Problem

The inventors, as a result of intensive study to develop an artificial plasma expander and a hemorrhagic shock resuscitation fluid with excellent safety and effectiveness, discovered that polyoxazoline-bound albumin, in which polyoxazoline that is a water-soluble polymer is covalently bound to the surface of albumin, functions as an artificial plasma expander and a hemorrhagic shock resuscitation fluid that are immunologically inactive, even in a case where the polyoxazoline-bound albumin is administered to heterogeneous animals, and can achieve the above-described purpose.

The polyoxazoline is a non-ionic (non-charged) water-soluble polymer consisting of a pseudopolypeptide structure that has high biocompatibility and is non-immunogenic. The polyoxazoline can exhibit many of the desirable properties of PEG while avoiding some of its drawbacks. The following is a list of notable characteristics of polyoxazoline (drawbacks of PEG in parentheses): (I) Polyoxazoline can be easily synthesized by the ring-opening polymerization of oxazoline, and various side-chain substituted derivatives can be easily prepared (PEG is difficult to be polymerized and has no side chain); (II) Polyoxazoline generates no peroxide (PEG generates peroxides); (III) Polyoxazoline has low viscosity (PEG has high viscosity at high concentration); (IV) Polyoxazoline is stable at room temperature (PEG is stable at low temperature but unstable at room temperature); and (V) Polyoxazoline resolves in the body and is easily removed (PEG may accumulate). The inventors discovered that polyoxazoline may be an alternative material of PEG, as a compound that modifies albumin.

That is, a polyoxazoline-bound albumin of this disclosure includes an albumin as a core and a polyoxazoline as a shell, which is covalently bound to the albumin via a cross-linker.

In the polyoxazoline-bound albumin of this disclosure, it is desirable that the albumin has a binding site to the cross-linker, and the binding site is ricin, primary amine at protein terminus, or cysteine.

In the polyoxazoline-bound albumin of this disclosure, it is desirable that the polyoxazoline has a binding site to the cross-linker, and the binding site is a terminal hydroxyl group or a terminal amino group of the polyoxazoline represented by the following General Formula (1):

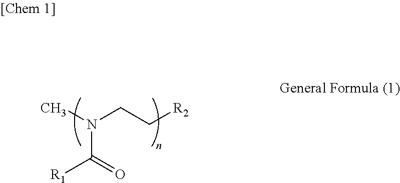

[in General Formula (1): R₁ represents a hydrocarbon group having 1 to 8 carbon atoms; R₂ represents a hydroxyl group, an amino group, or —NH—(CH₂)₂—OH; and n represents the number of repeating monomer units].

In the polyoxazoline-bound albumin of this disclosure, it is desirable that the covalent binding via the cross-linker includes the following Structure (1):

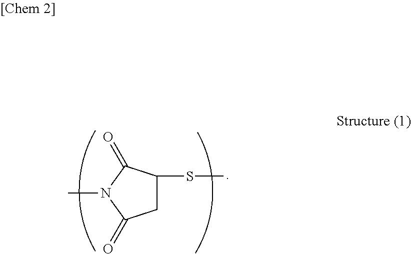

In the polyoxazoline-bound albumin of this disclosure, it is desirable that the covalent binding via the cross-linker includes a structure derived from a maleimide group introducing agent.

In the polyoxazoline-bound albumin of this disclosure, it is desirable that the maleimide group introducing agent contains at least one selected from the group consisting of a compound represented by the following General Formula (2) or a compound represented by the following General Formula (3):

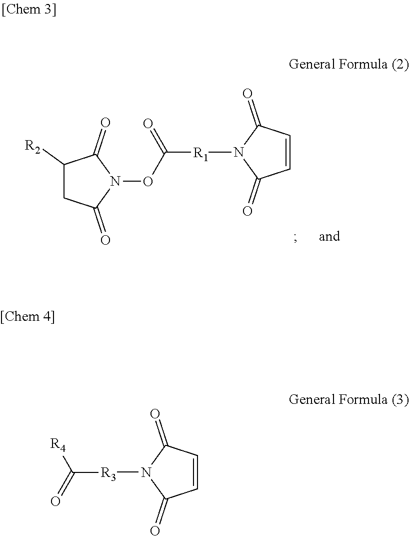

[in General Formula (2): R₂ represents a hydrogen atom or SO₃⁻Na⁺; and R₁ represents any one of the following General Formula (4), General Formula (5), Chemical Formula (1), or Chemical Formula (2), and in General Formula (3): R₃ represents the following General Formula (4); and R₄ represents OH or Cl]:

[Chem 5]

[symbol]

CH₂ [symbol] ₙ General Formula (4)

[in General Formula (4), n represents an integer of 1 to 10];

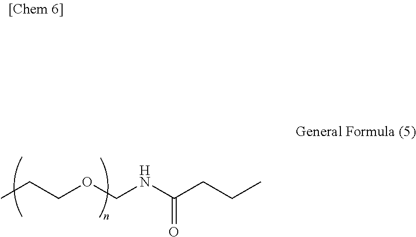

[in General Formula (5), n represents an integer of 2, 4, 6, 8, 10, or 12];

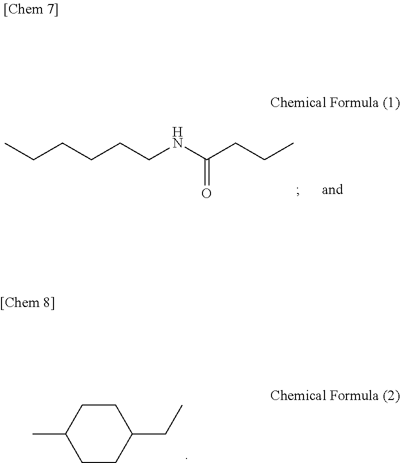

In the polyoxazoline-bound albumin of this disclosure, it is desirable that the covalent binding via the cross-linker includes the following Structure (2):

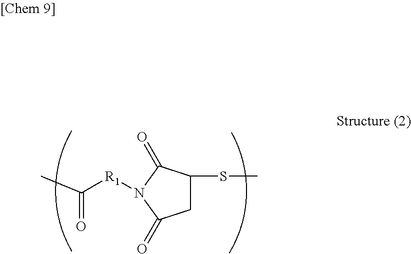

[in Structure (2), R₁ represents any one of the following General Formula (4), General Formula (5), Chemical Formula (1), or Chemical Formula (2)]:

[Chem 10]

[symbol]

CH₂ [symbol] ₙ General Formula (4)

[in General Formula (4), n represents an integer of 1 to 10];

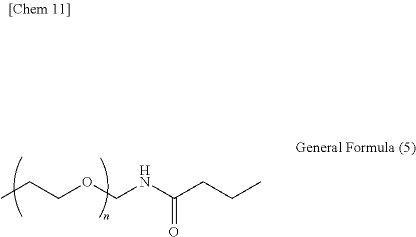

[in General Formula (5), n represents an integer of 2, 4, 6, 8, 10, or 12];

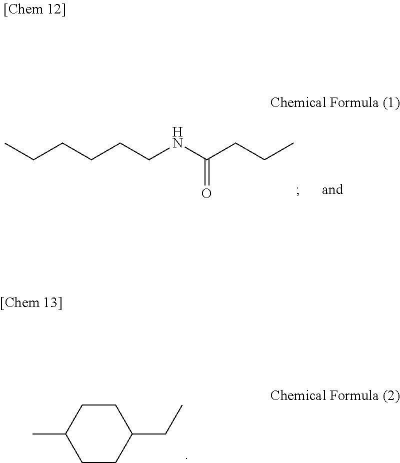

In the polyoxazoline-bound albumin of this disclosure, it is desirable that the covalent binding via the cross-linker further includes a structure derived from a thiol group introducing agent, and the thiol group introducing agent is at least one compound selected from the group consisting of a compound represented by the following Chemical Formula (3), a compound represented by the following General Formula (6), or a compound represented by the following General Formula (7):

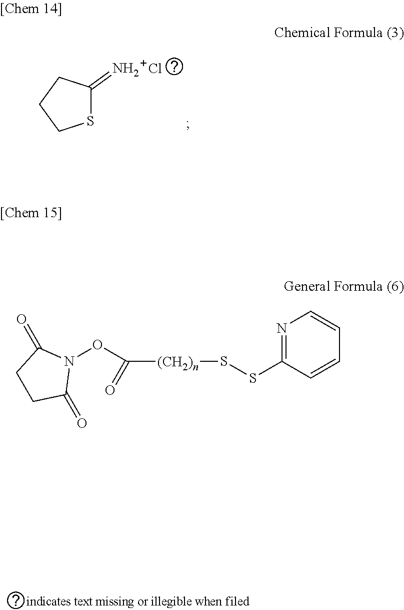

[in General Formula (6), n represents an integer of 1 to 10]; and

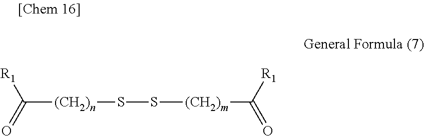

[in General Formula (7): R₁ represents OH or Cl; and n and m each represent an integer of 1 to 10].

In the polyoxazoline-bound albumin of this disclosure, it is desirable that the covalent binding via the cross-linker includes the following Structure (3) or Structure (4):

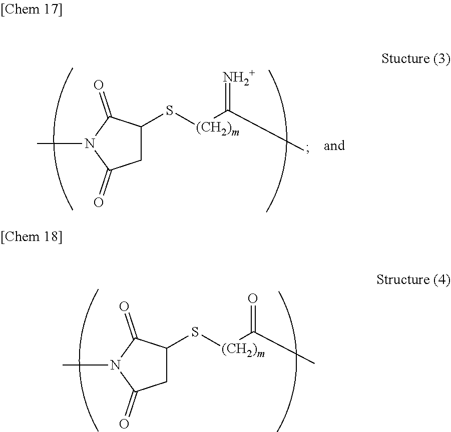

[in Structures (3) and (4), m represents an integer of 1 to 10].

In the polyoxazoline-bound albumin of this disclosure, it is desirable that the polyoxazoline has a weight-average molecular weight of 500 to 100,000 Dalton.

An artificial plasma expander of this disclosure contains the polyoxazoline-bound albumin.

A hemorrhagic shock resuscitation fluid of this disclosure contains the polyoxazoline-bound albumin.

### Advantageous Effect

This disclosure can provide new polyoxazoline-bound albumin, artificial plasma expander, and hemorrhagic shock resuscitation fluid that have high biocompatibility and are easily prepared (synthesized).

### BRIEF DESCRIPTION OF THE DRAWINGS

In the accompanying drawings:

FIG. 1 is a drawing of an example of polyoxazoline-bound albumin of this disclosure;

FIG. 2 is a graph that indicates the production quantities of anti-PSA IgM antibodies in a pig albumin (PSA) administrated group, a polyethylene glycol-bound pig albumin (PEG(5k)-eSM-PSA) administrated group, and a polyoxazoline-bound pig albumin (POx(5k)-eSM-PSA) administrated group;

FIG. 3A is a graph that indicates the number of red blood cells (RBCs) in blood in which polyoxazoline-bound pig albumin (POx(5k)-eSM-PSA) is mixed;

FIG. 3B is a graph that indicates the number of white blood cells (WBCs) in the blood in which polyoxazoline-bound pig albumin (POx(5k)-eSM-PSA) is mixed;

FIG. 3C is a graph that indicates the number of platelets (PLTs) in the blood in which polyoxazoline-bound pig albumin (POx(5k)-eSM-PSA) is mixed;

FIG. 4A is a graph that indicates the mean arterial pressures (MAPs) in a polyoxazoline-bound pig albumin (POx(5k)-eSM-PSA) administrated group and a hydroxyethyl starch (HES; Voluven infusion) administrated group after 50% of blood removal; and

FIG. 4B is a graph that indicates the heart rates (HRs) in the polyoxazoline-bound pig albumin (POx(5k)-eSM-PSA) administrated group and the hydroxyethyl starch (HES; Voluven infusion) administrated group after 50% of blood removal.

### DETAILED DESCRIPTION

In the following, an embodiment of this disclosure will be specifically exemplified and described, with reference to drawings, as necessary.

### Polyoxazoline-bound Albumin

Polyoxazoline-bound albumin of this embodiment has at least albumin as a core and polyoxazoline as a shell and further has other sites, as necessary. The albumin and the polyoxazoline are covalently bound to one another via a cross-linker.

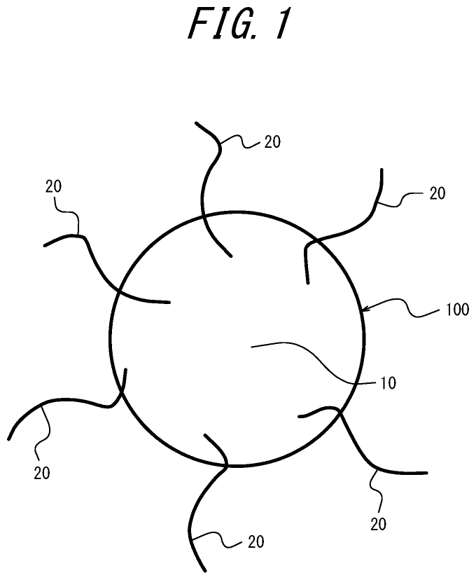

For example, as illustrated in FIG. 1, a polyoxazoline-bound albumin 100 of this embodiment may have an albumin 10 as a core and six polyoxazolines 20 as shells. In FIG. 1, the albumin 10 and the polyoxazolines 20 are covalently bound to one another via a cross-linker.

### Albumin

The albumin is a simple protein with a molecular weight of about 66500 Dalton.

In the polyoxazoline-bound albumin of this embodiment, polyoxazoline may be bound to albumin via a cross-linker.

The albumin can be appropriately selected according to the purpose without particular limitation, and albumin purified from serum derived from vertebrate including human can be used. The albumin is desirably at least one selected from the group consisting of human albumin, pig albumin, cow albumin, horse albumin, canine albumin, feline albumin, and recombinant albumin. These may be used singly or in combinations of two or more. Among these, pig albumin and cow albumin are preferable in terms of ensuring raw materials. Specific pathogen-free (SPF) pig-derived albumin is particularly preferable from the viewpoint of safety. The recombinant albumin can be easily produced by protein synthesis (culture).

### Human Albumin

The human albumin includes albumin purified from human-derived serum and can be appropriately selected according to the purpose without particular limitation.

### Pig Albumin

The pig albumin includes albumin purified from pig-derived serum and can be appropriately selected according to the purpose without particular limitation. The SPF pig-derived albumin includes albumin purified from SPF pig-derived serum.

### Cow Albumin

The cow albumin includes albumin purified from cow-derived serum and can be appropriately selected according to the purpose without particular limitation.

### Horse Albumin

The horse albumin includes albumin purified from horse-derived serum and can be appropriately selected according to the purpose without particular limitation.

### Canine Albumin

The canine albumin includes albumin purified from canine-derived serum and can be appropriately selected according to the purpose without particular limitation.

### Feline Albumin

The feline albumin includes albumin purified from feline-derived serum and can be appropriately selected according to the purpose without particular limitation.

### Recombinant Albumin

The recombinant albumin can be appropriately selected according to the purpose without particular limitation so long as it is produced by general gene recombination operation, culture operation, and the like.

### Cross-linker

Examples of the cross-linker include a cross-linker containing a maleimide group introducing agent and a cross-linker containing a thiol group introducing agent and specifically include the maleimide group introducing agent and the thiol group introducing agent. The cross-linker may be used singly or in combinations of two or more.

### Maleimide Group Introducing Agent

The maleimide group introducing agent can be appropriately selected according to the purpose without particular limitation so long as it is a reagent that can introduce a maleimide group into albumin or polyoxazoline, a compound having a maleimide group is preferably, and at least one compound selected from the group consisting of a bifunctional compound in which one terminal is a succinimidyl group and the other terminal is a maleimide group as in General Formula (2) below and a compound represented by General Formula (3) below is preferable from the viewpoint of reaction efficiency.

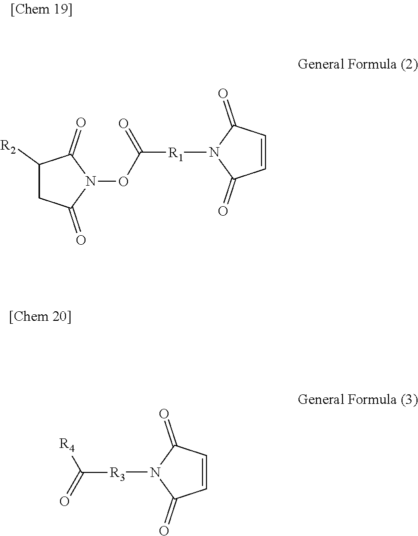

In General Formula (2), R₂ represents any one of a hydrogen atom or SO₃⁻Na⁺, and R₁ represents any one of General Formula (4) below, General Formula (5) below, Chemical Formula (1) below, or Chemical Formula (2) below.

In General Formula (3), R₃ represents General Formula (4) below, and R₄ represents OH or Cl.

[Chem 21]

[symbol]

CH₂ [symbol] ₙ General Formula (4)

In General Formula (4), n represents an integer of 1 to 10.

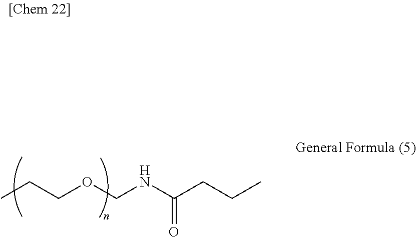

In General Formula (5), n represents an integer of 2, 4, 6, 8, 10, or 12.

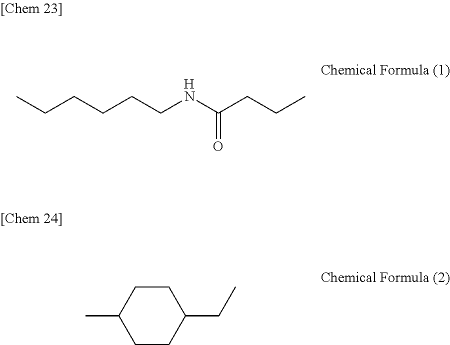

In a case where the maleimide group is introduced using a compound represented by General Formula (3) above in which R₄ is OH, it is preferable that the maleimide group is introduced into an NH₂ terminal or an OH terminal of albumin or polyoxazoline using a condensing agent together. Examples of the condensing agent preferably include N,N′-dicyclohexylcarbodiimide (DCC) and 1-ethyl-3-(3-dimethylaminopropyl) carbodiimide hydrochloride (EDC). The compound represented by General Formula (3) above in which R₄ is OH may be used, for example, after converting OH of R₄ to Cl through reaction with a compound such as thionyl chloride and oxalyl chloride.

As the above maleimide group introducing agent, a compound represented by General Formula (2) above or General Formula (3) above is preferable for introducing the maleimide group into an NH₂ group of ricin residue of albumin, an NH₂ group of primary amine at protein terminus, and a terminal NH₂ group of polyoxazoline, and a compound represented by General Formula (3) above is preferable for introducing the maleimide group into a terminal OH group of polyoxazoline. These may be used singly or in combinations of two or more.

For example, by reacting a succinimidyl group in the maleimide group introducing agent with an amino group (NH₂ group) of ricin residue of albumin or an amino group (NH₂ group) at protein terminus, the maleimide group can be introduced into the amino group (—NH₂) of ricin residue or the amino group (NH₂ group) at protein terminus.

Examples of a method for introducing the maleimide group include stirring albumin and the maleimide group introducing agent at 0° C. to 30° C. for 0.5 hours to 10 hours.

### Thiol Group Introducing Agent

The thiol group introducing agent can be appropriately selected according to the purpose without particular limitation so long as it is a reagent that can introduce a thiol group into albumin or polyoxazoline. From the viewpoint of reaction efficiency, the thiol group introducing agent is preferably at least one compound selected from the group consisting of a compound represented by Chemical Formula (3) below (2-Iminothiolane hydrochloride), a compound represented by General Formula (6) below, or a compound represented by General Formula (7) below.

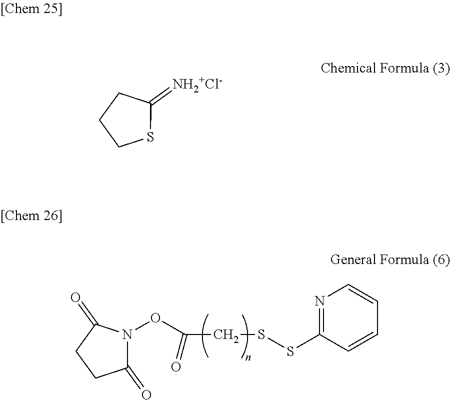

In General Formula (6), n represents an integer of 1 to 10. From the viewpoint of ease of crosslinking reaction, n is preferably 2 to 5, further preferably 2 to 3, and particular preferably n=2.

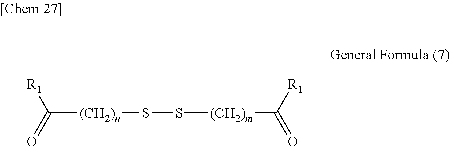

In General Formula (7), R₁ represents OH or Cl, and n and m each represent an integer of 1 to 10. Each R₁ may be the same or different. R₁ is preferably OH, and it is more preferable that two of R₁ are both OH. n and m may be the same or different. From the viewpoint of ease of crosslinking reaction, n and m are each preferably 2 to 5, further preferably 2 to 3, and particular preferably n=m=2.

In a case where the thiol group is introduced using a compound represented by General Formula (7) above in which R₁ is OH, it is preferable that the thiol group is introduced into an NH₂ terminal or an OH terminal of albumin or polyoxazoline using a condensing agent together. Examples of the condensing agent preferably include N,N-Dicyclohexylcarbodiimide (DCC) and 1-ethyl-3-(3-dimethylaminopropyl) carbodiimide hydrochloride (EDC). The compound represented by General Formula (7) above in which R₁ is OH may be used after, for example, converting OH of R₁ to Cl through reaction with a compound such as thionyl chloride and oxalyl chloride.

As the above thiol group introducing agent, a compound represented by Chemical Formula (3) above, a compound represented by General Formula (6) above, or a compound represented by General Formula (7) above is preferable for introducing the thiol group into an NH₂ group of ricin residue of albumin, an NH₂ group of primary amine at protein terminus, and a terminal NH₂ group of polyoxazoline, and a compound represented by General Formula (7) above is preferable for introducing the thiol group into a terminal OH group of polyoxazoline. These may be used singly or in combinations of two or more.

### Polyoxazoline

The polyoxazoline is a non-ionic water-soluble polymer consisting of a pseudopolypeptide structure that has high biocompatibility and is non-immunogenic. The polyoxazoline can be synthesized with high volume, introduce various functional groups, and be safely used for animals. The polyoxazoline can exhibit many of the excellent properties of PEG while avoiding some of its drawbacks. Further, the polyoxazoline is discharged from kidney without accumulating in the tissue in the body.

The above polyoxazoline may be used singly or in combinations of two or more.

The polyoxazoline includes a compound represented by General Formula (1) below.

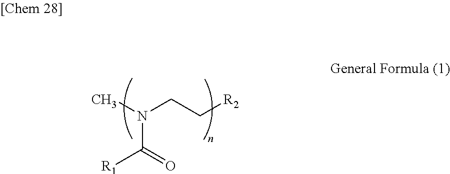

In General Formula (1), R₁ represents a hydrocarbon group having 1 to 8 carbon atoms and is preferably a methyl group, an ethyl group, a propyl group, or an isopropyl group. R₂ represents a hydroxyl group, an amino group, or —NH—(CH₂)₂—OH. n represents the number of repeating monomer units.

Here, the binding site to the cross-linker at the polyoxazoline is desirably a terminal hydroxyl group or a terminal amino group of polyoxazoline represented by General Formula (1) above.

The polyoxazoline may be a homopolymer or a heteropolymer.

The weight-average molecular weight of the polyoxazoline is preferably 500 to 100,000 Dalton.

### Method of Producing Polyoxazoline-bound Albumin

A method of producing the polyoxazoline-bound albumin of this embodiment includes a method of causing reaction using at least an albumin derivative derived from the albumin and a polyoxazoline derivative derived from the polyoxazoline.

### Albumin Derivative

The albumin derivative is desirably at least one selected from the group consisting of maleimide group-introduced albumin, thiol group-introduced albumin, and non-modified albumin.

Among these, in a case where terminal thiol group polyoxazoline is used as a polyoxazoline derivative, the maleimide group-introduced albumin is desirably used as an albumin derivative.

The maleimide group-introduced albumin is desirably maleimide group-introduced albumin obtained such that the above maleimide group introducing agent (for example, the compound represented by General Formula (2)) binds to the ricin residue (NH₂ group) of albumin or the amino group (NH₂ group) at protein terminus. The maleimide group-introduced albumin can be obtained by, for example, a method of stirring albumin and the maleimide group introducing agent at 0° C. to 30° C. for 0.5 hours to 10 hours.

In a case where terminal maleimide group polyoxazoline is used as a polyoxazoline derivative, the thiol group-introduced albumin or the non-modified albumin is desirably used as an albumin derivative.

The thiol group-introduced albumin is desirably thiol group-introduced albumin obtained such that the above thiol group introducing agent (for example, 2-Iminothiolane hydrochloride) binds to the amino group (NH₂ group) of the ricin residue of albumin or the amino group (NH₂ group) at protein terminus. The thiol group-introduced albumin can be obtained by, for example, a method of stirring albumin and the thiol group introducing agent such as 2-Iminothiolane hydrochloride at 0° C. to 30° C. for 0.5 hours to 10 hours.

Reductant-treated albumin may be used as the non-modified albumin.

### Polyoxazoline Derivative

The polyoxazoline derivative is desirably at least one selected from the group consisting of the terminal thiol group polyoxazoline and the terminal maleimide group polyoxazoline, and more desirably at least one selected from compounds represented by General Formulas (8) to (13) below.

Among these, in a case where the albumin derivative is maleimide group-introduced albumin, the terminal thiol group polyoxazoline is desirable, and in a case where the albumin derivative is thiol group-introduced albumin or non-modified albumin, the terminal maleimide group polyoxazoline is desirable.

The polyoxazoline derivative can be used, for example, as a cross-linker when polyoxazoline is introduced into a compound (for example, a compound having a thiol group at the terminal, a compound having a maleimide group at the terminal).

### Terminal Thiol Group Polyoxazoline

The terminal thiol group polyoxazoline can be appropriately selected according to the purpose without particular limitation. Examples of the terminal thiol group polyoxazoline include compounds represented by General Formulas (8) to (10) below. These may be used singly or in combinations of two or more. The weight-average molecular weight of the terminal thiol group polyoxazoline is preferably 500 to 100,000 Dalton.

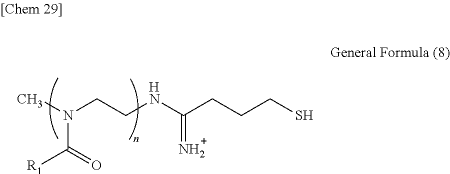

In General Formula (8), n represents the number of repeating monomer units. R₁ represents any one of a methyl group, an ethyl group, a propyl group, and an isopropyl group.

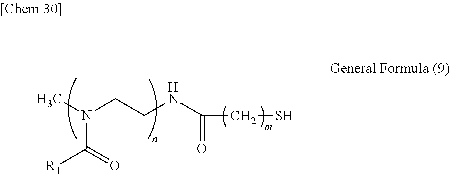

In General Formula (9), n represents the number of repeating monomer units, and m represents an integer of 1 to 10. R₁ represents any one of a methyl group, an ethyl group, a propyl group, and an isopropyl group.

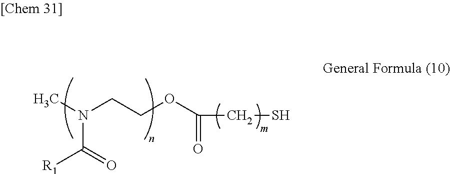

In General Formula (10), n represents the number of repeating monomer units, and m represents an integer of 1 to 10. R₁ represents any one of a methyl group, an ethyl group, a propyl group, and an isopropyl group.

Each structure of the repeating units in the terminal thiol group polyoxazoline may be the same or different.

The terminal thiol group polyoxazoline can be used as a cross-linker that adds polyoxazoline to a compound having a maleimide group at the terminal (for example, maleimide group-modified protein).

The terminal thiol group polyoxazoline can be obtained by, for example, reacting polyoxazoline with a thiol group introducing agent such as a compound in General Formula (7) above (for example, reaction through stirring at 25° C. for 1 hour to 96 hours). The mixing ratio may be 2 to 20 mol of the thiol group introducing agent such as the compound in General Formula (7) above to 1 mol of polyoxazoline. After the reaction, treatment with a reductant and purification by centrifugation, filter filtration, or gel filtration may be performed.

The structure of the terminal thiol group polyoxazoline to be obtained can be analyzed by ¹H-NMR or the like.

### Terminal Maleimide Group Polyoxazoline

The terminal maleimide group polyoxazoline can be appropriately selected according to the purpose without particular limitation, and examples of the terminal maleimide group polyoxazoline include compounds represented by General Formulas (11) to (13) below. These may be used singly or in combinations of two or more. The weight-average molecular weight of the terminal maleimide group polyoxazoline is preferably 500 to 100,000 Dalton.

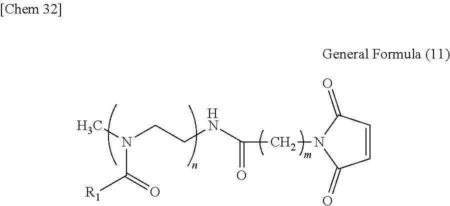

In General Formula (11), n represents the number of repeating monomer units, and m represents an integer of 1 to 10. R₁ represents any one of a methyl group, an ethyl group, a propyl group, and an isopropyl group.

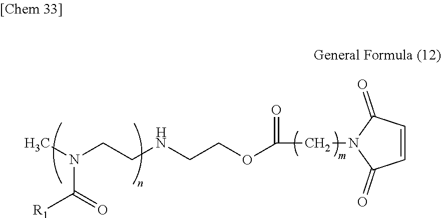

In General Formula (12), n represents the number of repeating monomer units, and m represents an integer of 1 to 10. R₁ represents any one of a methyl group, an ethyl group, a propyl group, and an isopropyl group.

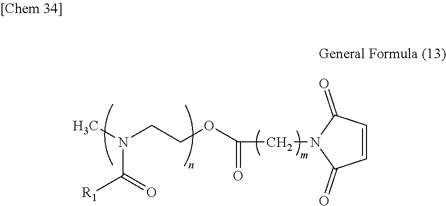

In General Formula (13), n represents the number of repeating monomer units, and m represents an integer of 1 to 10. R₁ represents any one of a methyl group, an ethyl group, a propyl group, and an isopropyl group.

Each structure of the repeating units in the terminal maleimide group polyoxazoline may be the same or different.

The terminal maleimide group polyoxazoline can be used as a cross-linker that adds polyoxazoline to a compound having a SH group at the terminal (for example, cysteine residue in protein, thiol group-modified protein).

The terminal maleimide group polyoxazoline can be obtained by, for example, reacting polyoxazoline with a maleimide group introducing agent such as a compound in General Formula (3) above (for example, reaction through stirring at 25° C. for 1 hour to 96 hours when R₄ is Cl, without change, and when R₄ is OH, by adding the condensing agent). The mixing ratio may be 2 to 20 mol of the maleimide group introducing agent such as the compound in General Formula (3) above to 1 mol of polyoxazoline. After the reaction, purification by centrifugation, filter filtration, or gel filtration may be performed.

The structure of the terminal maleimide group polyoxazoline to be obtained can be analyzed by ¹H-NMR or the like.

In General Formulas (8) to (13), any compound in which R₁ is a methyl group tends to indicate higher water solubility than PEG, and any compound in which R₁ is a propyl group tends to indicate reduced solubility to water by heating. Therefore, any compound in which R₁ is an ethyl group is preferable in that it has a good balance of hydrophilic and hydrophobic properties.

In General Formulas (8) to (13), any compound in which m is minimum, 2 with high chemical stable is preferable.

The method of producing the polyoxazoline-bound albumin more specifically includes, for example, methods of (a) to (c) below.

### Production Example (a) of Polyoxazoline-bound Albumin

By reacting the terminal thiol group polyoxazoline with the maleimide group-introduced albumin, the thiol group in the terminal thiol group polyoxazoline forms covalent binding with the maleimide group in the maleimide group-introduced albumin.

Examples of the method for introducing the polyoxazoline include stirring the maleimide group-introduced albumin and the terminal thiol group polyoxazoline at 0° C. to 30° C. for 1 hour to 72 hours.

### Production Example (b) of Polyoxazoline-bound Albumin

By reacting the terminal maleimide group polyoxazoline and the thiol group-introduced albumin, the maleimide group in the terminal maleimide group polyoxazoline forms covalent binding with the thiol group in the thiol group-introduced albumin.

Examples of the method for introducing the polyoxazoline include stirring the thiol group-introduced albumin and the terminal maleimide group polyoxazoline at 0° C. to 30° C. for 1 hour to 72 hours.

### Production Example (c) of Polyoxazoline-bound Albumin

By reacting the terminal maleimide group polyoxazoline with the non-modified albumin, the maleimide group in the terminal maleimide group polyoxazoline forms covalent binding with cysteine in the non-modified albumin.

Examples of the method for introducing the terminal maleimide group polyoxazoline include stirring the non-modified albumin and the terminal maleimide group polyoxazoline at 0° C. to 30° C. for 1 hour to 72 hours.

### Characteristics of Polyoxazoline-bound Albumin

In the polyoxazoline-bound albumin of this embodiment, the binding site to the cross-linker at the albumin is desirably ricin, primary amine at protein terminus, or cysteine.

In the polyoxazoline-bound albumin of this embodiment, the binding site to the cross-linker at the polyoxazoline is desirably a terminal hydroxyl group or a terminal amino group of polyoxazoline represented by General Formula (1) above.

The binding site to the terminal thiol group polyoxazoline at the maleimide group-introduced albumin is desirably the introduced maleimide group. The binding site to the terminal maleimide group polyoxazoline at the thiol group-introduced albumin is desirably the introduced thiol group. The binding site to the terminal maleimide group polyoxazoline at the non-modified albumin is desirably cysteine residue.

In the polyoxazoline-bound albumin of this embodiment, binding via the cross-linker preferably includes a structure derived from the maleimide group introducing agent and/or the thiol group introducing agent and more preferably includes a structure derived from only the maleimide group introducing agent and the thiol group introducing agent.

The above binding via the cross-linker preferably includes Structure (1) below:

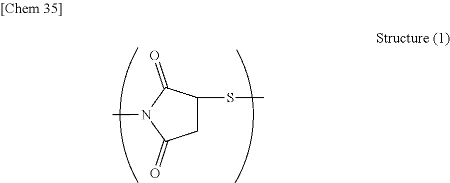

more preferably has Structure (2), Structure (3), or Structure (4) below:

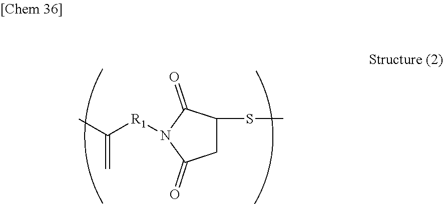

[in Structure (2), R₁ represents any one of General Formula (4), General Formula (5) above, Chemical Formula (1), or Chemical Formula (2) above];

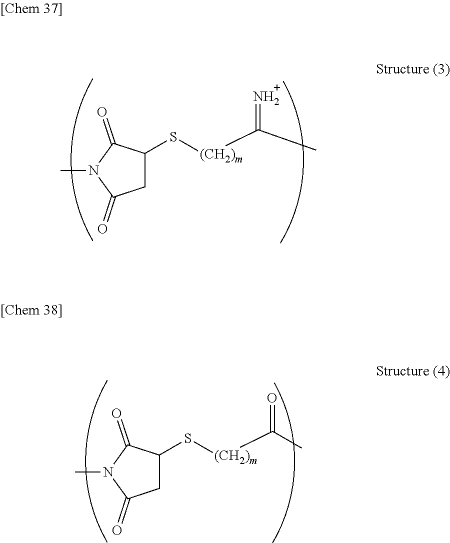

[in Structures (3) and (4), m represents an integer of 1 to 10]; and further preferably has Structure (5) or Structure (6) below:

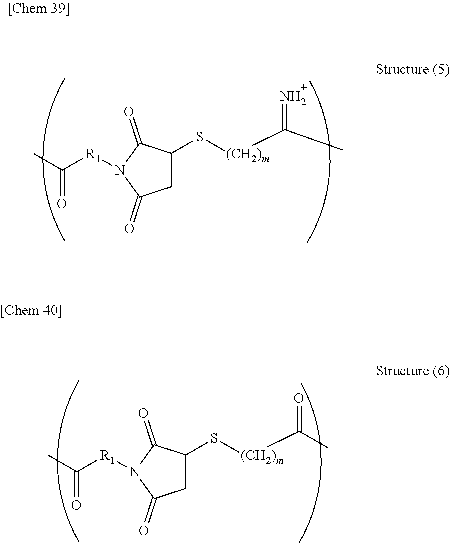

[in Structure (5) and Structure (6), R₁ represents any one of General Formula (4), General Formula (5) above, Chemical Formula (1), or Chemical Formula (2) above, and m represents an integer of 1 to 10].

The polyoxazoline-bound albumin of this embodiment has a clear three-dimensional structure although its synthesis and the like are easy. The mean particle size of the polyoxazoline-bound albumin is preferably 8 to 30 nm, and more preferably 10 to 20 nm.

The binding number of polyoxazoline to core albumin of the polyoxazoline-bound albumin of this embodiment is preferably 1 to 10.

A method of measuring the binding number of polyoxazoline to core albumin in the polyoxazoline-bound albumin of this embodiment includes a method of measuring the dry weight of the polyoxazoline-bound albumin.

The polyoxazoline-bound albumin of this embodiment has higher colloid osmotic pressure than the non-modified albumin and produces an effect on maintenance of the circulating blood volume compared with the non-modified albumin with the same concentration, when being administered to the living body.

The polyoxazoline-bound albumin of this embodiment does not develop immunogenicity even when being administered to heterogeneous animals because the core albumin is surrounded by polyoxazoline.

The polyoxazoline-bound albumin of this embodiment does not evoke precipitation and aggregation even when being mixed with blood, which has high blood compatibility.

The polyoxazoline-bound albumin of this embodiment does not cause renal excretion and leakage from vascular endothelial cell when being administered into the living body, which has longer retention time in blood than the non-modified albumin. The polyoxazoline has high water solubility and excellent metabolism.

The polyoxazoline-bound albumin of this embodiment recovers the circulating blood volume to improve blood pressure when being administered into the living body in a hemorrhage shock state.

As described above, the polyoxazoline-bound albumin of this disclosure can function as unparalleled artificial plasma expander and hemorrhagic shock resuscitation fluid having biocompatibility (safety) and effectiveness.

### Artificial Plasma Expander

The artificial plasma expander of this embodiment contains the polyoxazoline-bound albumin of the above-described embodiment. The artificial plasma expander is a substance having colloid osmotic pressure, which functions as an alternative of albumin of an animal when being administered into its living body.

The above artificial plasma expander can be used, for example, as an alternative of albumin of vertebrate such as human, pig, cow, horse, dog, cat, monkey, and rabbit.

### Hemorrhagic Shock Resuscitation Fluid

The hemorrhagic shock resuscitation fluid of this embodiment contains the polyoxazoline-bound albumin of the above-described embodiment. The hemorrhagic shock resuscitation fluid is a substance having colloid osmotic pressure, which functions as an alternative of albumin of an animal when being administered into its living body.

The above hemorrhagic shock resuscitation fluid can be used, for example, as an alternative of albumin of vertebrate such as human, pig, cow, horse, dog, cat, monkey, and rabbit.

### EXAMPLES

The following specifically describes this disclosure based on examples, but this disclosure is not limited to these examples.

### Example 1

### Preparation Example 1: Preparation of Maleimide Group-introduced Pig Albumin (PSA-M)

The following operation was performed to introduce a maleimide group into pig albumin (PSA).

A sample bottle (8-mL volume) was charged with 119.8 mg of N-succinimidyl 3-Maleimidopropionate (SMP; FUJIFILM Wako Pure Chemical Corporation), and it was dissolved with 3 mL of dimethyl sulfoxide to prepare a 0.15 M SMP solution. Next, a one-neck eggplant flask (100-mL volume) was charged with 30 mL of pig albumin (1 mM), 3 mL of SMP solution was added thereto (N-succinimidyl 3-Maleimidopropionate/albumin (SMP/PSA)=15 (mol/mol)), and these materials were stirred at 25° C. for 1 hour. After filtering these materials with a filter (Merck Milipore; Millex-GP; 0.22 μm; PES), the solution was passed through a gel filtration column (GE HEALTHCARE JAPAN; Sephadex G-25 Superfine) equilibrated with phosphate buffered saline (PBS; pH: 7.4) to remove excess N-succinimidyl 3-Maleimidopropionate.

PBS was added to the obtained solution to fix the volume at 90 mL.

### Preparation Example 2: Preparation of Terminal Thiol Group Polyoxazoline (Weight-average Molecular Weight: 5,000 Da; POx(5k)-eSH)

The following operation was performed to synthesize terminal thiol group polyoxazoline (weight-average molecular weight: 5,000 Da; POx(5k)-eSH).

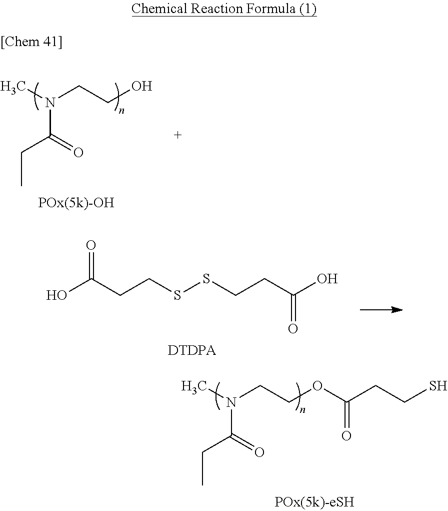

In Chemical Reaction Formula (1), n represents the number of repeating monomer units.

A three-neck eggplant flask (300-mL volume) was charged with 5 g of poly(2-ethyl-2-oxazoline) having a hydroxyl group at the terminal (weight-average molecular weight: 5,000 Da; POx(5k)-OH; Sigma-ALDRICH), 1.26 g of 3,3′-Dithiodipropionic Acid (DTDPA; Tokyo Chemical Industry Co., Ltd.), 1.26 g of N,N′-Dicyclohexylcarbodiimide (DCC; Tokyo Chemical Industry Co., Ltd.), and 160 mg of 4-Dimethylaminopyridine (DMAP; Tokyo Chemical Industry Co., Ltd.). After nitrogen aeration, 60 mL of tetrahydrofuran (FUJIFILM Wako Pure Chemical Corporation) was added thereto, and these materials were then stirred at 25° C. for 72 hours. The solvent in the reaction liquid was distilled with a rotatory evaporator (EYELA), and the white solid was dried using a vacuum pump. 50 mL of pure water was added thereto, and the precipitates were removed by centrifugation. After the pH was adjusted to 7 using a 5 M NaOH solution (FUJIFILM Wako Pure Chemical Corporation), 1.85 g of dithiothreitol (DTT; FUJIFILM Wako Pure Chemical Corporation) was added thereto. After nitrogen aeration for 5 minutes, these materials were stirred at 25° C. for 2 hours (dithiothreitol/polyoxazoline (DTT/POx(5k)-SH)=12 (mol/mol)). After filtering the solution with a filter (Merck Milipore; Millex-GP; 0.22 μm; PES), dialysis was performed using a dialysis membrane (molecular weight cut off: 3500 Da; Spectrum Laboratories) to remove unreacted materials.

After the obtained aqueous solution was frozen with liquid nitrogen, it was lyophilized under vacuum to identify the structure by ¹H NMR. The thiol concentration in the obtained polyoxazoline solution was determined using exchange reaction between the thiol group and the disulfide bond. 4,4′-Dithiopyridine (4,4′-DTP) reacts with a free thiol (SH) group to generate 4-Thiopyridinone (4-TP). Thus, the amount of the thiol group can be determined by adding 4,4′-Dithiopyridine (4,4′-DTP) to thiol group-introduced albumin and measuring the amount of 4-Thiopyridinone (4-TP) that is generated. When the introducing rate of the thiol group in terminal thiol group polyoxazoline (POx(5k)-eSH) was computed from the obtained concentration, it was about 96%.

### Preparation Example 3: Preparation of Polyoxazoline (Weight-average Molecular Weight: 5,000 Da)-bound Albumin (POx(5k)-eSM-PSA)

The following operation was performed to prepare polyoxazoline-bound albumin (POx(5k)-eSM-PSA) in which polyoxazoline (weight-average molecular weight: 5,000 Da) is bound.

A one-neck flask (300-mL volume) was charged with 90 mL of the maleimide group-introduced pig albumin solution (PSA-M; 333 μM) obtained in Preparation Example 1, and 60 mL of PBS solution (3.75 mM) of the terminal thiol group polyoxazoline (weight-average molecular weight: 5,000 Da; POx(5k)-eSH) obtained in Preparation Example 2 was added thereto, and these materials were then stirred at 25° C. for 24 hours (terminal thiol group polyoxazoline/maleimide group-introduced albumin (POx(5k)-eSH/PSA-M) =7.5 (mol/mol)).

The reaction liquid was subjected to cycle ultrafiltration (Merck; Pelicon XL cassette; molecular weight cut off: 100 kDa) to remove unreacted polyoxazoline. When the binding number of polyoxazoline to core albumin was computed by measuring the dry weight of the polyoxazoline-bound albumin, it was about 6 per PSA.

### Example 2

### Preparation Example 1: Preparation of Terminal Thiol Group Polyoxazoline (Weight-average Molecular Weight: 5,000 Da; POx(5k)-aSH)

The following operation was performed to synthesize terminal thiol group polyoxazoline (weight-average molecular weight: 5,000 Da; POx(5k)-aSH).

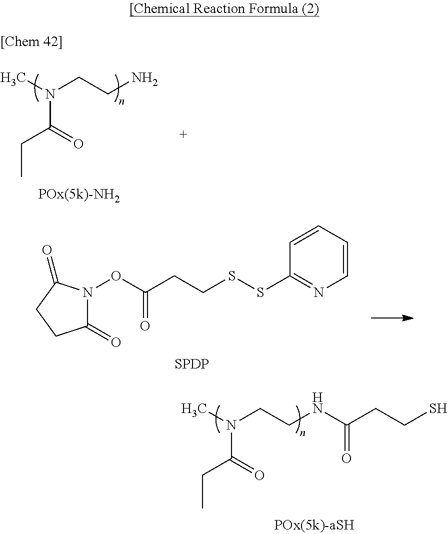

In Chemical Reaction Formula (2), n represents the number of repeating monomer units.

A two-neck eggplant flask (100-mL volume) was charged with 200 mg of poly(2-ethyl-2-oxazoline) having an amino group at the terminal (weight-average molecular weight: 5,000 Da; POx(5k)-NH₂; Sigma-ALDRICH) and 124 mg of N-succinimidyl 3-(2-pyridyldithio)propionate (SPDP; Tokyo Chemical Industry Co., Ltd.), and nitrogen aeration was then performed (N-succinimidyl 3-(2-pyridyldithio)propionate/polyoxazoline (SPDP/POx(5k)-NH₂)=10 (mol/mol)).

20 mL of dichloromethane (FUJIFILM Wako Pure Chemical Corporation) was added thereto, and these materials were then stirred at 25° C. for 15 hours. The solvent in the reaction liquid was distilled with a rotatory evaporator (EYELA), 12 mL of pure water and 124 mg of dithiothreitol (DTT; FUJIFILM Wako Pure Chemical Corporation) were added to the reaction liquid, and the reaction liquid was stirred at 25° C. for 2 hours (dithiothreitol/polyoxazoline (DTT/POx(5k)-NH₂)=20 (mol/mol)). After the precipitates were removed by centrifugation, the supernatant was filtered with a filter (Merck Milipore; Millex-GP; 0.22 μm; PES), and the reaction liquid was passed through a gel filtration column (GE HEALTHCARE JAPAN; PD-10) equilibrated with pure water to remove unreacted materials.

After the obtained aqueous solution was frozen with liquid nitrogen, it was lyophilized under vacuum to identify the structure by ¹H NMR. When the thiol concentration in the obtained polyoxazoline solution was determined using exchange reaction between the thiol group and the disulfide bond and the introducing rate of the thiol group in the terminal thiol group polyoxazoline (POx(5k)-aSH) was computed, it was about 95%.

### Preparation Example 2: Preparation of Polyoxazoline (Weight-average Molecular Weight: 5,000 Da)-bound Albumin (POx(5k)-aSM-PSA)

The following operation was performed to prepare polyoxazoline-bound albumin (POx(5k)-aSM-PSA) in which polyoxazoline (weight-average molecular weight: 5,000 Da) is bound.

A one-neck flask (30-mL volume) was charged with 9.0 mL of the maleimide group-introduced albumin solution (PSA-M; 333 μM) obtained in Preparation Example 1 in Example 1, and 6.0 mL of PBS solution (3.75 mM) of the terminal thiol group polyoxazoline (weight-average molecular weight: 5,000 Da; POx(5k)-aSH) obtained in Preparation Example 1 in Example 2 was added thereto, and these materials were then stirred at 25° C. for 24 hours (terminal thiol group polyoxazoline/maleimide group-introduced albumin (POx(5k)-aSH/PSA-M)=7.5 (mol/mol)).

The reaction liquid was subjected to cycle ultrafiltration (Merck; Pelicon XL cassette; molecular weight cut off: 100 kDa) to remove unreacted polyoxazoline. When the binding number of polyoxazoline to core albumin was computed by measuring the dry weight of the polyoxazoline-bound albumin, it was about 6 per PSA.

### Example 3

### Preparation Example 1: Preparation of Terminal Thiol Group Polyoxazoline (Weight-average Molecular Weight: 10,000 Da; POx(10k)-eSH)

Terminal thiol group polyoxazoline (weight-average molecular weight: 10,000 Da; POx(10k)-eSH) was prepared according to the same method as Preparation Example 2 in Example 1, except for using poly(2-ethyl-2-oxazoline) having a hydroxy group at the terminal (weight-average molecular weight: 10,000 Da; POx(10k)-OH), instead of poly(2-ethyl-2-oxazoline) having a hydroxy group at the terminal (weight-average molecular weight: 5,000 Da; POx(5k)-OH), in Preparation Example 2 in Example 1. When the thiol concentration in the obtained polyoxazoline solution was determined using exchange reaction between the thiol group and the disulfide bond and the introducing rate of the thiol group in the terminal thiol group polyoxazoline (POx(10k)-eSH) was computed, it was about 95%.

### Preparation Example 2: Preparation of Polyoxazoline (Weight-average Molecular Weight: 10,000 Da)-bound Albumin (POx(10k)-eSM-PSA)

Polyoxazoline-bound albumin (POx(10k)-eSM-PSA) was prepared according to the same method as Preparation Example 3 in Example 1, except for using the terminal thiol group polyoxazoline (weight-average molecular weight: 10,000 Da; POx(10k)-eSH) obtained in Preparation Example 1 in Example 3, instead of terminal thiol group polyoxazoline (weight-average molecular weight: 5,000 Da; POx(5k)-eSH), in Preparation Example 3 in Example 1. When the binding number of polyoxazoline to core albumin was computed by measuring the dry weight of the polyoxazoline-bound albumin, it was about 6 per PSA.

### Example 4

### Preparation Example 1: Preparation of Maleimide Group-introduced Human Albumin (HSA-M)

Maleimide group-introduced human albumin (HSA-M) was prepared according to the same method as Preparation Example 1 in Example 1, except for using human albumin, instead of pig albumin, in Preparation Example 1 in Example 1.

### Preparation Example 2: Preparation of Polyoxazoline (Weight-average Molecular Weight: 5,000 Da)-bound Albumin (POx(5k)-eSM-HSA)

Polyoxazoline-bound albumin (POx(5k)-eSM-HSA) was prepared according to the same method as Preparation Example 3 in Example 1, except for using the maleimide group-introduced human albumin (HSA-M) obtained in Preparation Example 1 in Example 4, instead of maleimide group-introduced pig albumin (PSA-M), in Preparation Example 3 in Example 1. When the binding number of polyoxazoline to core albumin was computed by measuring the dry weight of the polyoxazoline-bound albumin, it was about 6 per HSA.

### Example 5

### Preparation Example 1: Preparation of Polyoxazoline (Weight-average Molecular Weight: 10,000 Da)-bound Albumin (POx(10k)-eSM-HSA)

Polyoxazoline-bound albumin (POx(10k)-eSM-HSA) was prepared according to the same method as Preparation Example 3 in Example 1, except for using the terminal thiol group polyoxazoline (weight-average molecular weight: 10,000 Da; POx(10k)-eSH) obtained in Preparation Example 1 in Example 3, instead of terminal thiol group polyoxazoline (weight-average molecular weight: 5,000 Da; POx(5k)-eSH), and further using the maleimide group-introduced human albumin (HSA-M) obtained in Preparation Example 1 in Example 4, instead of maleimide group-introduced pig albumin (PSA-M), in Preparation Example 3 in Example 1. When the binding number of polyoxazoline to core albumin was computed by measuring the dry weight of the polyoxazoline-bound albumin, it was about 6 per HSA.

### Example 6

### Preparation Example 1: Preparation of Maleimide Group-introduced Cow Albumin (BSA-M)

Maleimide group-introduced cow albumin (BSA-M) was prepared according to the same method as Preparation Example 1 in Example 1, except for using cow albumin, instead of pig albumin, in Preparation Example 1 in Example 1.

### Preparation Example 2: Preparation of Polyoxazoline (Weight-average Molecular Weight: 5,000 Da)-bound Albumin (POx(5k)-eSM-BSA)

Polyoxazoline-bound albumin (POx(5k)-eSM-BSA) was prepared according to the same method as Preparation Example 3 in Example 1, except for using the maleimide group-introduced cow albumin (BSA-M) obtained in Preparation Example 1 in Example 6, instead of maleimide group-introduced pig albumin (PSA-M), in Preparation Example 3 in Example 1. When the binding number of polyoxazoline to core albumin was computed by measuring the dry weight of the polyoxazoline-bound albumin, it was about 6 per BSA.

### Example 7

### Preparation Example 1: Preparation of Polyoxazoline (Weight-average Molecular Weight: 10,000 Da)-bound Albumin (POx(10k)-eSM-BSA)

Polyoxazoline-bound albumin (POx(10k)-eSM-BSA) was prepared according to the same method as Preparation Example 3 in Example 1, except for using the terminal thiol group polyoxazoline (weight-average molecular weight: 10,000 Da; POx(10k)-eSH) obtained in Preparation Example 1 in Example 3, instead of terminal thiol group polyoxazoline (weight-average molecular weight: 5,000 Da; POx(5k)-eSH), and further using the maleimide group-introduced cow albumin (BSA-M) obtained in Preparation Example 1 in Example 6, instead of maleimide group-introduced pig albumin (PSA-M), in Preparation Example 3 in Example 1. When the binding number of polyoxazoline to core albumin was computed by measuring the dry weight of the polyoxazoline-bound albumin, it was about 6 per BSA.

### Example 8

### Preparation Example 1: Preparation of Maleimide Group-introduced Pig Albumin (PSA-MC)

Maleimide group-introduced pig albumin (PSA-MC) was prepared according to the same method as Preparation Example 1 in Example 1, except for using N-succinimidyl 4-(N-Maleimidomethyl)cyclohexanecarboxylate (SMCC; FUJIFILM Wako Pure Chemical Corporation), instead of N-succinimidyl 3-Maleimidopropionate (SMP; FUJIFILM Wako Pure Chemical Corporation), in Preparation Example 1 in Example 1.

### Preparation Example 2: Preparation of Polyoxazoline (Weight-average Molecular Weight: 5,000 Da)-bound Albumin (POx(5k)-eSMC-PSA)

Polyoxazoline-bound albumin (POx(5k)-eSMC-PSA) was prepared according to the same method as Preparation Example 3 in Example 1, except for using the maleimide group-introduced pig albumin (PSA-MC) obtained in Preparation Example 1 in Example 8, instead of maleimide group-introduced pig albumin (PSA-M), in Preparation Example 3 in Example 1. When the binding number of polyoxazoline to core albumin was computed by measuring the dry weight of the polyoxazoline-bound albumin, it was about 6 per PSA.

### Example 9

### Preparation Example 1: Preparation of Polyoxazoline (Weight-average Molecular Weight: 10,000 Da)-bound Albumin (POx(10k)-eSMC-PSA)

Polyoxazoline-bound albumin (POx(10k)-eSMC-PSA) was prepared according to the same method as Preparation Example 3 in Example 1, except for using the terminal thiol group polyoxazoline (weight-average molecular weight: 10,000 Da; POx(10k)-eSH) obtained in Preparation Example 1 in Example 3, instead of terminal thiol group polyoxazoline (weight-average molecular weight: 5,000 Da; POx(5k)-eSH), and further using the maleimide group-introduced pig albumin (PSA-MC) obtained in Preparation Example 1 in Example 8, instead of maleimide group-introduced pig albumin (PSA-M), in Preparation Example 3 in Example 1. When the binding number of polyoxazoline to core albumin was computed by measuring the dry weight of the polyoxazoline-bound albumin, it was about 6 per PSA.

### Example 10

### Preparation Example 1: Preparation of Thiol Group-introduced Pig Albumin (PSA-SH)

The following operation was performed to introduce a thiol group into pig albumin.

A microtube (1.5-mL volume) was charged with 13.8 mg of 2-Iminothiolane hydrochloride (2-IT; FUJIFILM Wako Pure Chemical Corporation), and it was diluted with 1 mL of phosphate buffered saline (PBS; pH: 7.4) to prepare a 0.1 M 2-Iminothiolane solution. Next, a one-neck eggplant flask (10-mL volume) was charged with 1 mL of pig albumin (1 mM), 400 μL of the 2-Iminothiolane solution was added thereto (2-Iminothiolane/albumin (2-IT/PSA)=40 (mol/mol)), and these materials were then stirred at 25° C. for 3 hours. The solution was passed through a gel filtration column (GE HEALTHCARE JAPAN; Sephadex G-25 Superfine) equilibrated with phosphate buffered saline (PBS; pH: 7.4) to remove excess 2-Iminothiolane (2IT).

20 mL of the obtained solution was put into a centrifugal concentrator (Merck, Amicon Ultra-15; molecular weight cut off: 10 kDa) to be concentrated to 2.5 mL by centrifugation (0.4 mM).

### Preparation Example 2: Preparation of Terminal Maleimide Group Polyoxazoline (Weight-average Molecular Weight: 5,000 Da; POx(5k)-eM)

The following operation was performed to synthesize terminal maleimide group polyoxazoline (weight-average molecular weight: 5,000 Da; POx(5k)-eM).

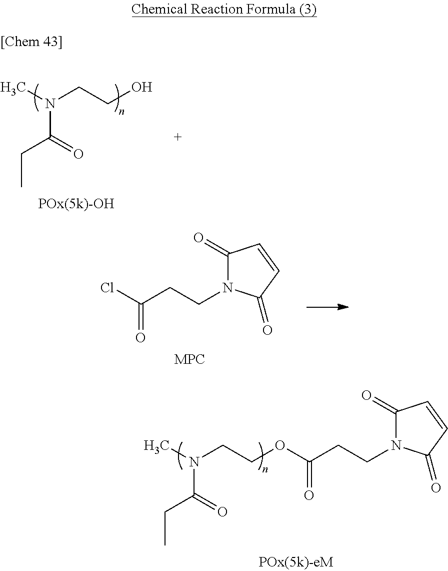

In Chemical Reaction Formula (3), n represents the number of repeating monomer units.

A two-neck eggplant flask (50-mL volume) was charged with 120 mg of poly(2-ethyl-2-oxazoline) having a hydroxyl group at the terminal (weight-average molecular weight: 5,000 Da; POx(5k)-OH; Sigma-ALDRICH) and 45 mg of 3-maleimidopropanoly chloride (MPC) prepared by reacting 3-maleimidopropionic acid with thionyl chloride, and nitrogen aeration was then performed (3-maleimidopropanoly chloride/polyoxazoline (MPC/POx(5k)-OH)=10 (mol/mol)).

5 mL of dichloromethane (FUJIFILM Wako Pure Chemical Corporation) and 66 μL of triethylamine (FUJIFILM Wako Pure Chemical Corporation) were added thereto, and these materials were then stirred at 25° C. for 18 hours. The solvent in the reaction liquid was distilled with a rotatory evaporator (EYELA), 3 mL of pure water was added to the reaction liquid, and the reaction liquid was then well stirred. After the precipitates were removed by centrifugation, the supernatant was filtered with a filter (Merck Milipore; Millex-GP; 0.22 μm; PES), and the reaction liquid was purified using a gel filtration column (GE HEALTHCARE JAPAN; Sephadex G-25 Superfine) equilibrated with pure water.

After the obtained aqueous solution was frozen with liquid nitrogen, it was lyophilized under vacuum to identify the structure by ¹H NMR.

### Preparation Example 3: Preparation of Polyoxazoline (Weight-average Molecular Weight: 5,000 Da)-bound Albumin (POx(5k)-eMS-PSA)

The following operation was performed to prepare polyoxazoline-bound albumin (POx(5k)-eMS-PSA) in which polyoxazoline (weight-average molecular weight: 5,000 Da) is bound.

A one-neck flask (5-mL volume) was charged with 625 μL of the thiol group-introduced albumin solution (PSA-SH; 0.4 mM) obtained in Preparation Example 1, 12.5 mg of the terminal maleimide group polyoxazoline (weight-average molecular weight: 5,000 Da; POx(5k)-eM) obtained in Preparation Example 2 was added thereto, and these materials were then stirred at 25° C. for 14 hours (terminal maleimide group polyoxazoline/thiol group-introduced albumin (POx(5k)-eM/PSA-SH)=10 (mol/mol)).

The reaction liquid was passed through a gel filtration column (GE HEALTHCARE JAPAN; Superdex 200 p.g.) equilibrated with phosphate buffered saline (PBS; pH: 7.4) to remove unreacted polyoxazoline. When the binding number of polyoxazoline to core albumin was computed by measuring the dry weight of the polyoxazoline-bound albumin, it was about 6 per PSA.

### Example 11

### Preparation Example 1: Preparation of Terminal Maleimide Group Polyoxazoline (Weight-average Molecular Weight: 5,000 Da; POx(5k)-aM)

The following operation was performed to synthesize terminal maleimide group polyoxazoline (weight-average molecular weight: 5,000 Da; POx(5k)-aM).

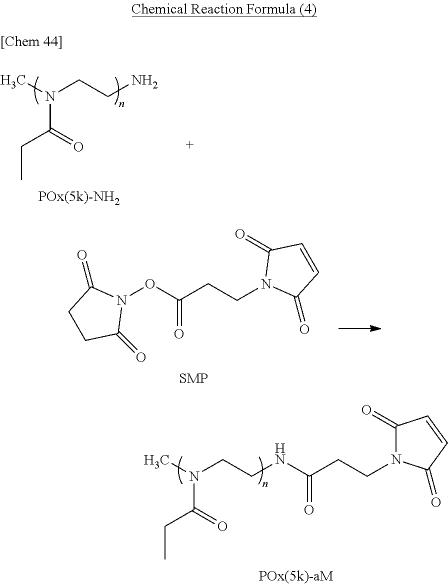

In Chemical Reaction Formula (4), n represents the number of repeating monomer units.

A two-neck eggplant flask (50-mL volume) was charged with 50 mg of poly(2-ethyl-2-oxazoline) having an amino group at the terminal (weight-average molecular weight: 5,000 Da; POx(5k)-NH₂; Sigma-ALDRICH) and 26.5 mg of N-succinimidyl 3-maleimidopropionate (SMP; FUJIFILM Wako Pure Chemical Corporation), and nitrogen aeration was then performed (N-succinimidyl 3-maleimidopropionate/polyoxazoline (SMP/POx(5k)-NH₂)=10 (mol/mol)).

5 mL of dichloromethane (FUJIFILM Wako Pure Chemical Corporation) was added thereto, and these materials were then stirred at 25° C. for 18 hours. The solvent in the reaction liquid was distilled with a rotatory evaporator (EYELA), 2 mL of pure water was added to the reaction liquid, and the reaction liquid was then well stirred. After the precipitates were removed by centrifugation, the supernatant was filtered with a filter (Merck Milipore; Millex-GP; 0.22 μm; PES), and the reaction liquid was then passed through a gel filtration column (GE HEALTHCARE JAPAN; Sephadex G-25 Superfine) equilibrated with pure water to remove unreacted N-succinimidyl 3-Maleimidopropionate (SMP).

After the obtained aqueous solution was frozen with liquid nitrogen, it was lyophilized under vacuum to identify the structure by ¹H NMR.

### Preparation Example 2: Preparation of Polyoxazoline (Weight-average Molecular Weight: 5,000 Da)-bound Albumin (POx(5k)-aMS-PSA)

Polyoxazoline (weight-average molecular weight: 5,000 Da)-bound albumin (POx(5k)-aMS-PSA) was prepared according to the same method as Preparation Example 3 in Example 10, except for using the terminal maleimide group polyoxazoline (weight-average molecular weight: 5,000 Da; POx(5k)-aM) obtained in Preparation Example 1 in Example 11, instead of terminal maleimide group polyoxazoline (weight-average molecular weight: 5,000 Da; POx(5k)-eM), in Preparation Example 3 in Example 10. When the binding number of polyoxazoline to core albumin was computed by measuring the dry weight of the polyoxazoline-bound albumin, it was about 6 per PSA.

### Example 12

### Preparation Example 1: Preparation of Terminal Maleimide Group Polyoxazoline (Weight-average Molecular Weight: 10,000 Da; POx(10k)-eM)

Terminal maleimide group polyoxazoline (weight-average molecular weight: 10,000 Da; POx(10k)-eM) was prepared according to the same method as Preparation Example 2 in Example 10, except for using poly(2-ethyl-2-oxazoline) having a hydroxy group at the terminal (weight-average molecular weight: 10,000 Da; POx(10k)-OH), instead of poly(2-ethyl-2-oxazoline) having a hydroxy group at the terminal (weight-average molecular weight: 5,000 Da; POx(5k)-OH), in Preparation Example 2 in Example 10.

### Preparation Example 2: Preparation of Polyoxazoline (Weight-average Molecular Weight: 10,000 Da)-bound Albumin (POx(10k)-eMS-PSA)

Polyoxazoline-bound albumin (POx(10k)-eMS-PSA) was prepared according to the same method as Preparation Example 3 in Example 10, except for using the terminal maleimide group polyoxazoline (weight-average molecular weight: 10,000 Da; POx(10k)-eM) obtained in Preparation Example 1 in Example 12, instead of terminal maleimide group polyoxazoline (weight-average molecular weight: 5,000 Da; POx(5k)-eM), in Preparation Example 3 in Example 10. When the binding number of polyoxazoline to core albumin was computed by measuring the dry weight of the polyoxazoline-bound albumin, it was about 6 per PSA.

### Example 13

### Preparation Example 1: Preparation of Thiol Group-introduced Human Albumin (HSA-M)

Thiol group-introduced human albumin (HSA-SH) was prepared according to the same method as Preparation Example 1 in Example 10, except for using human albumin, instead of pig albumin, in Preparation Example 1 in Example 10.

### Preparation Example 2: Preparation of Polyoxazoline (Weight-average Molecular Weight: 5,000 Da)-bound Albumin (POx(5k)-eMS-HSA)

Polyoxazoline-bound albumin (POx(5k)-eMS-HSA) was prepared according to the same method as Preparation Example 3 in Example 10, except for using the thiol group-introduced human albumin (HSA-SH) obtained in Preparation Example 1 in Example 13, instead of thiol group-introduced pig albumin (PSA-SH), in Preparation Example 3 in Example 10. When the binding number of polyoxazoline to core albumin was computed by measuring the dry weight of the polyoxazoline-bound albumin, it was about 6 per HSA.

### Example 14

### Preparation Example 1: Preparation of Polyoxazoline (Weight-average Molecular Weight: 10,000 Da)-bound Albumin (POx(10k)-eMS-HSA)

Polyoxazoline-bound albumin (POx(10k)-eMS-HSA) was prepared according to the same method as Preparation Example 3 in Example 10, except for using the terminal maleimide group polyoxazoline (weight-average molecular weight: 10,000 Da; POx(10k)-eM) obtained in Preparation Example 1 in Example 12, instead of terminal maleimide group polyoxazoline (weight-average molecular weight: 5,000 Da; POx(5k)-eM), and further using the thiol group-introduced human albumin (HSA-SH) obtained in Preparation Example 1 in Example 13, instead of thiol group-introduced pig albumin (PSA-SH), in Preparation Example 3 in Example 10. When the binding number of polyoxazoline to core albumin was computed by measuring the dry weight of the polyoxazoline-bound albumin, it was about 6 per HSA. Table 1 presents the summary of Examples 1 to 14.

TABLE 1

| | Polyoxazoline- | Albumin | Polyoxazoline | Binding number |
|---|---|---|---|---|
| | bound albumin | derivative | derivative | of polyoxazoline |
| Example 1 | POx(5k)-eSM-PSA | PSA-M | POx(5k)-eSH | About 6 per PSA |
| Example 2 | POx(5k)-aSM-PSA | PSA-M | POx(5k)-aSH | About 6 per PSA |
| Example 3 | POx(10k)-eSM-PSA | PSA-M | POx(10k)-eSH | About 6 per PSA |
| Example 4 | POx(5k)-eSM-HSA | HSA-M | POx(5k)-eSH | About 6 per HSA |
| Example 5 | POx(10k)-eSM-HSA | HSA-M | POx(10k)-eSH | About 6 per HSA |
| Example 6 | POx(5k)-eSM-BSA | BSA-M | POx(5k)-eSH | About 6 per BSA |
| Example 7 | POx(10k)-eSM-BSA | BSA-M | POx(10k)-eSH | About 6 per BSA |
| Example 8 | POx(5k)-eSMC-PSA | PSA-MC | POx(5k)-eSH | About 6 per PSA |
| Example 9 | POx(10k)-eSMC-PSA | PSA-MC | POx(10k)-eSH | About 6 per PSA |
| Example 10 | POx(5k)-eMS-PSA | PSA-SH | POx(5k)-eM | About 6 per PSA |
| Example 11 | POx(5k)-aMS-PSA | PSA-SH | POx(5k)-aM | About 6 per PSA |
| Example 12 | POx(10k)-eMS-PSA | PSA-SH | POx(10k)-eM | About 6 per PSA |
| Example 13 | POx(5k)-eMS-HSA | HSA-SH | POx(5k)-eM | About 6 per HSA |
| Example 14 | POx(10k)-eMS-HSA | HSA-SH | POx(10k)-eM | About 6 per HSA |

As presented in Table 1, it is found that polyoxazoline-bound albumin can be prepared from various albumin derivatives and polyoxazoline derivatives.

### Example 15

### Dynamic Light Scattering (DLS) Measurement

A phosphate buffered saline solution (PBS; pH: 7.4) of the polyoxazoline-bound albumin (POx(5k)-eSM-PSA) obtained in Preparation Example 3 in Example 1 was subjected to dynamic light scattering (DLS) measurement using a zeta potential, particle size, and molecular weight measurement system (Otsuka Electronics Co., Ltd.; ELSZ-2000). Non-modified pig albumin (PSA) was similarly tested.

The mean particle size of the non-modified pig albumin (PSA) was 8 nm. The mean particle size of the polyoxazoline-bound albumin (POx(5k)-eSM-PSA) was 13 nm. It was found that the binding of polyoxazoline to albumin increases the molecular size.

### Example 16

### Colloid Osmotic Pressure Measurement

A phosphate buffered saline solution (PBS; pH: 7.4; [PSA]=5 g/dL) of the polyoxazoline-bound albumin (POx(5k)-eSM-PSA) obtained in Preparation Example 3 in Example 1 was subjected to colloid osmotic pressure measurement using a colloid osmometer (OSMOMAT 050; Gomotec). The polyoxazoline-bound albumin (POx(10k)-eSM-PSA) obtained in Preparation Example 2 in Example 3 and non-modified pig albumin (PSA) were similarly tested.

The colloid osmotic pressure of the non-modified pig albumin (PSA; 5 g/dL) was 18 mmHg. The colloid osmotic pressure of the polyoxazoline-bound albumin (POx(5k)-eSM-PSA) was 36 mmHg. The colloid osmotic pressure of the polyoxazoline-bound albumin (POx(10k)-eSM-PSA) was 62 mmHg. It was found that the binding of polyoxazoline to albumin increases the colloid osmotic pressure.

### Example 17

### Preparation Example 1: Preparation of Polyethylene Glycol (Weight-average Molecular Weight: 5,000 Da)-bound Albumin (PEG(5k)-eSM-PSA)

The following operation was performed to prepare polyethylene glycol-bound albumin (PEG(5k)-eSM-PSA) in which polyethylene glycol (weight-average molecular weight: 5,000 Da) is bound.

A one-neck flask (5-mL volume) was charged with 1.7 mL of the maleimide group-introduced albumin solution (PSA-M; 333 μM) obtained in Preparation Example 1 in Example 1, 28 mg of terminal thiol group polyethylene glycol (weight-average molecular weight: 5,000 Da; PEG(5k)-SH; NOF CORPORATION) was added thereto, and these materials were stirred at 25° C. for 24 hours (terminal thiol group polyethylene glycol/maleimide group-introduced albumin (PEG(5k)-SH/PSA-M)=10 (mol/mol)).

The reaction liquid was subjected to cycle ultrafiltration (Merck; Pelicon XL cassette; molecular weight cut off: 100 kDa) to remove unreacted polyethylene glycol. When the binding number of polyethylene glycol to core albumin was computed by measuring the dry weight of the polyethylene glycol-bound albumin, it was about 8 per PSA.

### Immunogenicity Test Using Rat

From the tail vein of a Wister rat (male; 7 week-old; about 180 g), a phosphate buffered saline solution (PBS; pH: 7.4; [PSA]=5 g/dL) of albumin (PSA), a phosphate buffered saline solution (PBS; pH: 7.4; [PSA]=5 g/dL) of the polyoxazoline-bound albumin (POx(5k)-eSM-PSA) obtained in Preparation Example 3 in Example 1, and a phosphate buffered saline solution (PBS; pH: 7.4; [PSA]=5 g/dL) of the polyethylene glycol-bound albumin (PEG(5k)-eSM-PSA) obtained in Preparation Example 1 in Example 17 were each administered (200 mg-PSA/kg-rat). 0, 1, 2, 3, 4, 5, 6, 7, 14, 21, and 28 days later, 100 μL of blood was each collected from the tail vein, and the supernatant obtained by centrifugation was stored at −80° C. On or after 28 days, each supernatant was subjected to indirect ELISA measurement to determine the production quantity of IgM antibodies against PSA.

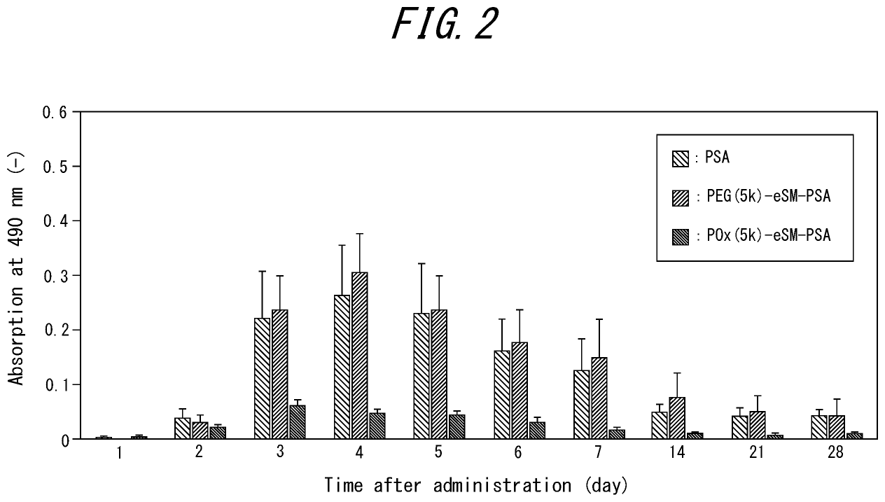

In the albumin (PSA) administrated group, the production of anti-PSA IgM antibodies was observed with a peak 4 days after administration. In the polyethylene glycol-bound albumin (PEG(5k)-eSM-PSA) administrated group, the production of anti-PSA IgM antibodies at the same level as the albumin (PSA) administrated group was observed. In contrast, the production of anti-PSA IgM antibodies in the polyoxazoline-bound albumin (POx(5k)-eSM-PSA) administrated group was significantly low (see FIG. 2). It was found that polyoxazoline binding exhibits excellent immunological stealth.

### Example 18

### Blood Compatibility Test

The blood was collected from a Wister rat (male; 7 week-old; about 230 g; Charls river) using a vacuum blood collection tube containing EDTA and mixed well with EDTA by inverted stirring. The obtained rat blood and a phosphate buffered saline solution (PBS; pH: 7.4; [PSA]=5 g/dL) of the polyoxazoline-bound albumin (POx(5k)-eSM-PSA) obtained in Preparation Example 3 in Example 1 were mixed such that the volume ratio of the polyoxazoline-bound albumin solution was 0, 10, 20, and 40% (total volume: 600 μL ). The sample was stood in a thermostat at 37° C. and fractionated by 50 μL 0 (immediately after mixing), 1, 2, 3, 4, 5, and 6 hours later, and the number of red blood cells (RBCs), the number of white blood cells (WBCs), and the number of platelets (PLTs) were measured (n=3) using a multiparameter hematology analyzer (pocH-100iV Diff; Sysmex).

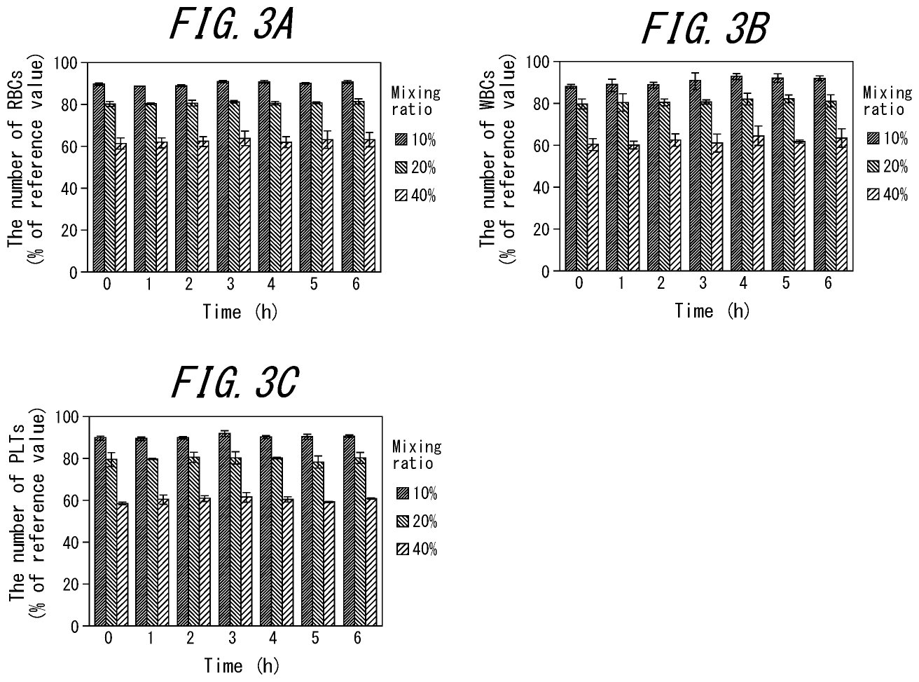

When the mixing ratio of the polyoxazoline-bound albumin solution was 10, 20, and 40%, each blood cell number was 90, 80, and 60% of the blood cell number (reference value) in the blood in which the polyoxazoline-bound albumin solution was not mixed (see FIGS. 3A to 3C). The blood cell number did not change 6 hours later, and it was found that the blood compatibility of the polyoxazoline-bound albumin was high.

### Example 19

### Measurement of Blood Retentivity in Rat

A Wister rat (male; 7 week-old; about 230 g; Charls river) anesthetized with Sevoflurane (Maruishi Pharmaceutical. Co., Ltd.) (5.0% in air) was fixed in a supine position on a thermal pad (DC Temperature controller; Brain Science Idea Co., Ltd.) under inhalation anesthesia with Sevoflurane (3.0 to 4.0% in air). A catheter (SP-31) was inserted into the right jugular vein by about 3 cm so that its tip could enter the right atrium. The opposite end of the catheter was subcutaneously passed to be fixed onto the back outer skin. A phosphate buffered saline solution (PBS; pH: 7.4; [PSA]=5 g/dL) of the polyoxazoline-bound albumin (POx(5k)-eSM-PSA) obtained in Preparation Example 3 in Example 1, which has been fluorescently labeled with Cy5.5, was administered from the right jugular vein (5% topload) (the dose was 5% of the circulating blood volume (56 mL/kg) of the rat (140 mg/kg-rat), fluorescent labelled : non-fluorescent labelled=1:9). The time 3 minutes after administration was set to 0 minutes, 200 μL of blood was each collected from the right jugular vein 0, 5, 15, and 30 minutes later and 1, 3, 6, 12, 18, and 24 hours later, and the serum component (100 μL) obtained by centrifugation (6,000 rpm; 5 min) was fractionated to be stored with refrigerated and light shielded. 20 μL of serum component, 12 μL of TritonX-100 PB solution, and 28 μL of PBS solution were mixed (serum component: 3 times dilution; TritonX-100: 1% (w/v)) and stood overnight under refrigerated and light shielded environment. This solution was put into a 3-mm micro quartz cell (minimum sample volume: 50 μL), and the fluorescence spectrum of the polyoxazoline-bound albumin fluorescently labeled with Cy5.5 was measured using a fluorescence spectrophotometer (JASCO FP-8300). The fluorescence intensity at 710 nm of serum collected at 0 minutes was set to 100%, and the blood disappearance half time (t_{1/2}) of the polyoxazoline-bound albumin was computed from the degree of reduction in fluorescence intensity. The polyoxazoline-bound albumin (POx(10k)-eSM-PSA) obtained in Preparation Example 2 in Example 3 and non-modified pig serum albumin (PSA) were similarly tested.

The blood disappearance half time (t_{1/2}) of the non-modified pig serum albumin (PSA) was 7.3 hours. The blood disappearance half time (t_{1/2}) of the polyoxazoline-bound albumin (POx(5k)-eSM-PSA) was 15.3 hours. The blood disappearance half time (t_{1/2}) of the polyoxazoline-bound albumin (POx(10k)-eSM-PSA) was 21.5 hours. It was found that the binding of polyoxazoline to albumin extends the blood disappearance half time (t_{1/2}).

### Example 20

### Effectiveness Evaluation by Rat 50% Hemorrhagic Shock Model

A Wister rat (male; 7 week-old; about 230 g; Charls river) anesthetized with Sevoflurane (Maruishi Pharmaceutical. Co., Ltd.) (5.0% in air) was fixed in a supine position on a thermal pad (DC Temperature controller; Brain Science Idea Co., Ltd.) under inhalation anesthesia with Sevoflurane (3.0% in air). A catheter (SP-31; inner diameter: 0.5 mm; outer diameter: 0.8 mm; Natsume Seisakusho Co., Ltd.) for blood pressure measurement and blood removal was inserted into the right carotid artery toward the center, and its opposite end was connected to a blood pressure measuring device (PAS-101; STARMEDICAL, Inc.). An identical catheter was inserted into the right jugular vein for sample administration. Tracheal cannula was performed, and respiratory care was performed using a ventilator. The hemorrhagic shock state was created by removing 50% of the total blood amount (56 mL/kg) from the arterial catheter (1 mL/min). The rat was resuscitated by administering (1 mL/min) a phosphate buffered saline solution (PBS; pH: 7.4; [PSA]=5 g/dL) (n=6) of the polyoxazoline-bound albumin (POx(5k)-eSM-PSA) prepared in Preparation Example 3 in Example 1 and a hydroxyethyl starch aqueous solution (Otsuka Pharmaceutical Co., Ltd.; Voluven infusion: 6%) (n=6) from the intravenous catheter 15 minutes later (the dose was equivalent to 30% of the total blood amount (56 mL/kg)).

The vital signs (mean arterial pressure (MAP), heart rate (HR), respiratory rate, and rectal temperature) were recorded at the following ten points of time: (1) before 50% blood removal, (2) immediately after 50% blood removal, (3) immediately before sample administration, (4) immediately after sample administration, (5) 5 minutes after administration, (6) 15 minutes after administration, (7) 30 minutes after administration, (8) 1 hour after administration, (9) 1.5 hours after administration, and (10) 2 hours after administration.

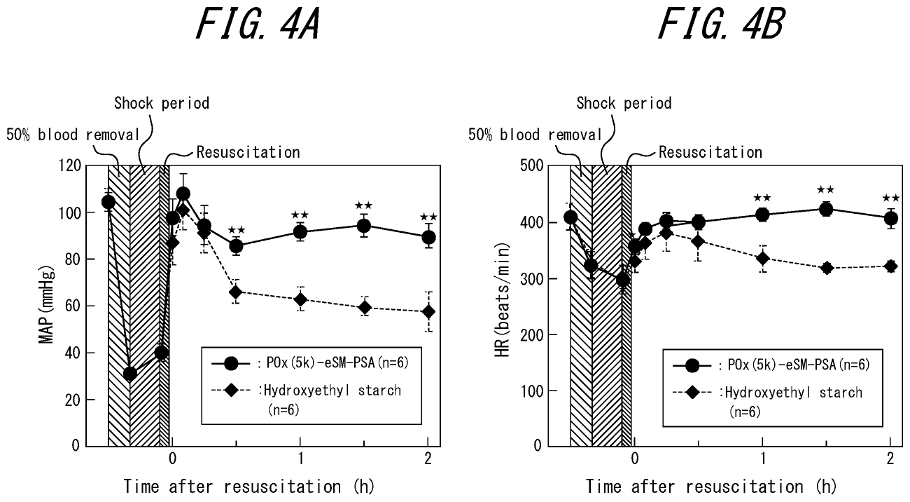

The mean arterial pressure (MAP) that had been about 100 mmHg decreased to about 30 mmHg after blood removal, increased by the administration of a polyoxazoline-bound albumin solution, and recovered to about 90 mmHg 2 hours after administration (see FIG. 4A; **p<0.01 versus hydroxyethyl starch). On the other hand, in the hydroxyethyl starch administrated group, the mean arterial pressure (MAP) increased only to about 60 mmHg 2 hours after administration. The heart rate (HR) that had been about 400 beats/min decreased to about 300 beats/min after blood removal, increased by the administration of the polyoxazoline-bound albumin solution, and recovered to about 400 beats/min 2 hours after administration (see FIG. 4B; *p<0.05, **p<0.01 versus hydroxyethyl starch). On the other hand, in the hydroxyethyl starch administrated group, the heart rate (HR) increased only to about 320 beats/min 2 hours after administration. It was found that the administration of the polyoxazoline-bound albumin solution was effective for resuscitation from the hemorrhagic shock state. Other vital signs and a blood gas parameter were recovered to their initial values by the administration of the polyoxazoline-bound albumin solution.

### Industrial Applicability

An artificial plasma expander using the polyoxazoline-bound albumin of this disclosure as an active ingredient can be used as a high-security plasma substitute even in a case of administration into the living body. The target is not limited to human, and the artificial plasma expander can be administered to animals (pets such as dogs and cats, livestock, etc.). The artificial plasma expander using the polyoxazoline-bound albumin of this disclosure as an active ingredient is administered in a case where hypoalbuminemia develops due to bleeding, an increase in permeability of the capillary, a decrease in albumin synthesis in the liver, excess excretion from the kidney or bowels, facilitation of metabolism, dilution by intraoperative infusion, or the like. Specifically, the artificial plasma expander can be expected to be used as a therapeutic agent such as in hemorrhagic shock, sepsis, cardiac surgery using a heart-lung machine, extracorporeal circulation with unstable hemodynamics, severe burn, pathological conditions such as pregnancy-induced hypertension, refractory ascites associated with hepatic cirrhosis, refractory edema, nephrotic syndrome associated with pulmonary edema, and protein-losing enteropathy.

### REFERENCE SIGNS LIST

100 polyoxazoline-bound albumin
10 albumin
20 polyoxazoline

1. A polyoxazoline-bound albumin comprising: an albumin as a core; and a polyoxazoline as a shell, the polyoxazoline being covalently bound to the albumin via a cross-linker.

2. The polyoxazoline-bound albumin according to claim 1, wherein the albumin has a binding site to the cross-linker, the binding site being ricin, primary amine at protein terminus, or cysteine.

3. The polyoxazoline-bound albumin according to claim 1, wherein the polyoxazoline has a binding site to the cross-linker, the binding site being a terminal hydroxyl group or a terminal amino group of the polyoxazoline represented by the following General Formula (1): 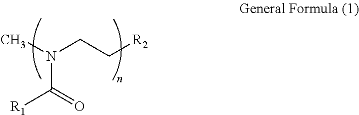
[in General Formula (1): R₁ represents a hydrocarbon group having 1 to 8 carbon atoms; R₂ represents a hydroxyl group, an amino group, or —NH—(CH₂)₂—OH; and n represents the number of repeating monomer units].

4. The polyoxazoline-bound albumin according to claim 1, wherein the covalent binding via the cross-linker includes the following Structure (1):

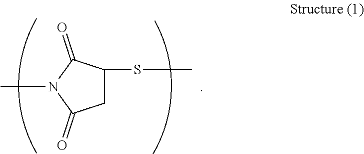

5. The polyoxazoline-bound albumin according to claim 1, wherein the covalent binding via the cross-linker includes a structure derived from a maleimide group introducing agent.

6. The polyoxazoline-bound albumin according to claim 5, wherein the maleimide group introducing agent contains at least one selected from the group consisting of a compound represented by the following General Formula (2) or a compound represented by the following General Formula (3): 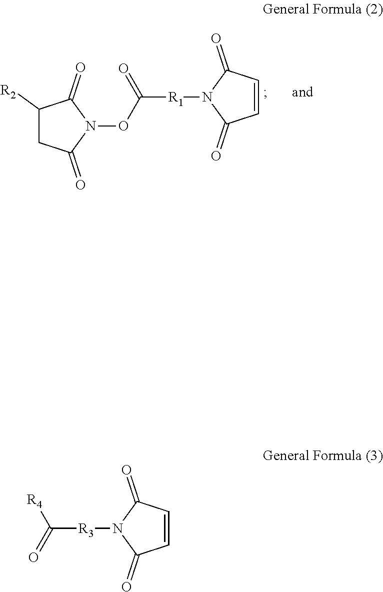
[in General Formula (2): R₂ represents a hydrogen atom or SO₃⁻Na⁺; and R₁ represents any one of the following General Formula (4), General Formula (5), Chemical Formula (1), or Chemical Formula (2), and in General Formula (3): R₃ represents the following General Formula (4); and R₄ represents OH or Cl]: [symbol]
CH₂ [symbol] ₙ General Formula (4)
[in General Formula (4), n represents an integer of 1 to 10]; 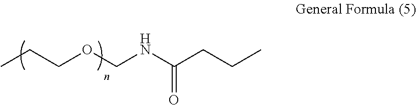
[in General Formula (5), n represents an integer of 2, 4, 6, 8, 10, or 12];

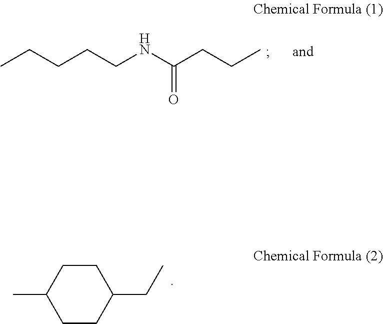

7. The polyoxazoline-bound albumin according to claim 1, wherein the covalent binding via the cross-linker includes the following Structure (2): 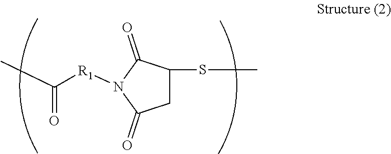
[in Structure (2), R₁ represents any one of the following General Formula (4), General Formula (5), Chemical Formula (1), or Chemical Formula (2)]: [symbol]
CH₂ [symbol] ₙ General Formula (4)
[in General Formula (4), n represents an integer of 1 to 10]; 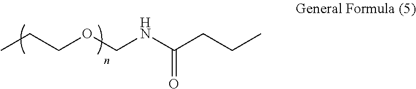
[in General Formula (5), n represents an integer of 2, 4, 6, 8, 10, or 12];

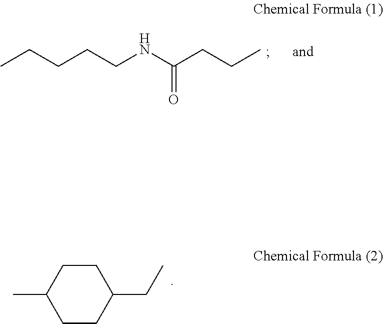

8. The polyoxazoline-bound albumin according to claim 5, wherein the covalent binding via the cross-linker further includes a structure derived from a thiol group introducing agent, and
the thiol group introducing agent is at least one compound selected from the group consisting of a compound represented by the following Chemical Formula (3), a compound represented by the following General Formula (6), or a compound represented by the following General Formula (7): 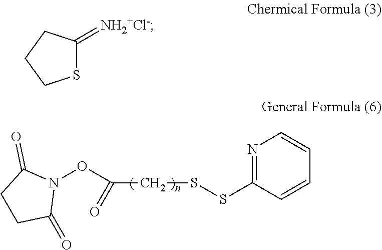
[in General Formula (6), n represents an integer of 1 to 10]; and 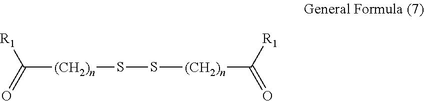
[in General Formula (7): R₁ represents OH or Cl; and n and m each represent an integer of 1 to 10].

9. The polyoxazoline-bound albumin according to claim 1, wherein the covalent binding via the cross-linker includes the following Structure (3) or Structure (4): 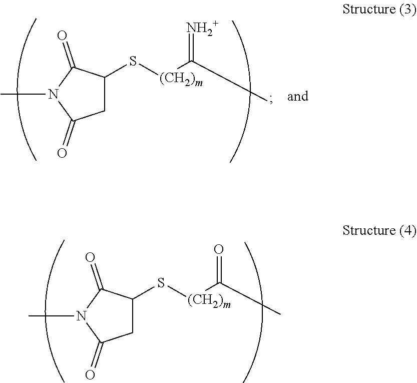
[in Structure (3) and Structure (4), m represents an integer of 1 to 10].

10. The polyoxazoline-bound albumin according to claim 1, wherein the polyoxazoline has a weight-average molecular weight of 500 to 100,000 Dalton.

11. An artificial plasma expander comprising the polyoxazoline-bound albumin according to claim 1.

12. A hemorrhagic shock resuscitation fluid comprising the polyoxazoline-bound albumin according to claim 1.

13. The polyoxazoline-bound albumin according to claim 2, wherein the polyoxazoline has a binding site to the cross-linker, the binding site being a terminal hydroxyl group or a terminal amino group of the polyoxazoline represented by the following General Formula (1): 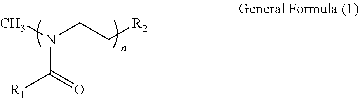
[in General Formula (1): R₁ represents a hydrocarbon group having 1 to 8 carbon atoms; R₂ represents a hydroxyl group, an amino group, or —NH—(CH₂)₂—OH; and n represents the number of repeating monomer units].

14. The polyoxazoline-bound albumin according to claim 2, wherein the covalent binding via the cross-linker includes the following Structure (1):

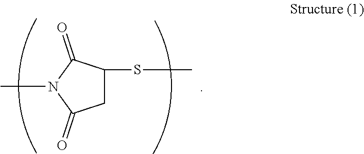

15. The polyoxazoline-bound albumin according to claim 2, wherein the covalent binding via the cross-linker includes a structure derived from a maleimide group introducing agent.

16. The polyoxazoline-bound albumin according to claim 2, wherein the covalent binding via the cross-linker includes the following Structure (2): 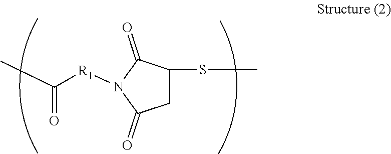
[in Structure (2), R₁ represents any one of the following General Formula (4), General Formula (5), Chemical Formula (1), or Chemical Formula (2)]: [symbol]
CH₂ [symbol] ₙ General Formula (4)
[in General Formula (4), n represents an integer of 1 to 10]; 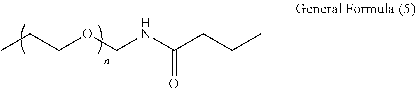
[in General Formula (5), n represents an integer of 2, 4, 6, 8, 10, or 12];

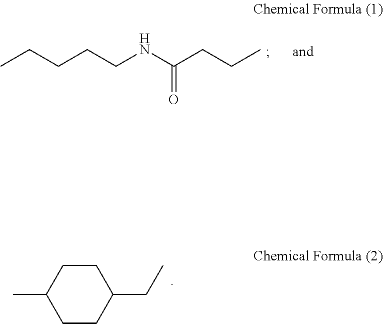

17. The polyoxazoline-bound albumin according to claim 2, wherein the covalent binding via the cross-linker includes the following Structure (3) or Structure (4): 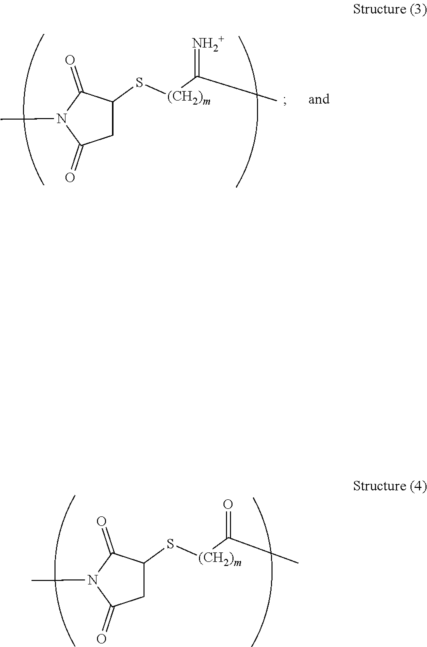
[in Structure (3) and Structure (4), m represents an integer of 1 to 10].

18. The polyoxazoline-bound albumin according to claim 2, wherein the polyoxazoline has a weight-average molecular weight of 500 to 100,000 Dalton.

19. An artificial plasma expander comprising the polyoxazoline-bound albumin according to claim 2.

20. A hemorrhagic shock resuscitation fluid comprising the polyoxazoline-bound albumin according to claim 2.
